SEQUENCE LISTING (10 sequences)

SEQ ID NO: 1 - protein, 141 aa, Artificial Sequence
  Construct design of neoantigenic construct #1
VPLQSLTGEVFAHASLFVHVAGGSATSQFKGYRRKSSLNGKGESSGGSAIYEVPKEMHENKQHLQKDFFGGSADREFQFFDHNLMESIKMGDPGGSGQTAIDAALTFVVDQDGGVHIGGSMPIGRIADCRVLWDYVYQLLS

SEQ ID NO: 2 - protein, 141 aa, Artificial Sequence
  Construct design of neoantigenic construct #2
VPLQSLTGEVFAHASLFVHVAGGSATSQFKGYRRKSSLNGKGESSGGSAIYEVPKEMHENKQHLQKDFFGGSADREFQFFDHNLMESIKMGDPGGSASPLKSIWSVLTPSPIKSTLGGGSMPIGRIADCRVLWDYVYQLLS

SEQ ID NO: 3 - protein, 189 aa, Artificial Sequence
  Neoantigenic construct #1
MKAIGGSKGGSKGGSKGSWGSKGGSKGGSKGGSKGGSAVPLQSLTGEVFAHASLFVHVAGGSATSQFKGYRRKSSLNGKGESSGGSAIYEVPKEMHENKQHLQKDFFGGSADREFQFFDHNLMESIKMGDPGGSGQTAIDAALTFVVDQDGGVHIGGSMPIGRIADCRVLWDYVYQLLSSGHHHHHHHH

SEQ ID NO: 4 - protein, 189 aa, Artificial Sequence
  Neoantigenic construct #2
MKAIGGSKGGSKGGSKGSWGSKGGSKGGSKGGSKGGSAVPLQSLTGEVFAHASLFVHVAGGSATSQFKGYRRKSSLNGKGESSGGSAIYEVPKEMHENKQHLQKDFFGGSADREFQFFDHNLMESIKMGDPGGSASPLKSIWSVLTPSPIKSTLGGGSMPIGRIADCRVLWDYVYQLLSSGHHHHHHHH

SEQ ID NO: 5 - protein, 189 aa, Artificial Sequence
  Wild-type antigenic construct #1
MKAIGGSKGGSKGGSKGSWGSKGGSKGGSKGGSKGGSAVPLQSLTGEVLAHASLFVHVAGGSATSQFKGYRRRSSLNGKGESSGGSAIYEVPKEMHGNKQHLQKDFFGGSADREFQFFDHHLMESIKMGDPGGSGQTAIDAALTSVVDQDGGVHIGGSMPIGRIADCRLLWDYVYQLLSSGHHHHHHHH

SEQ ID NO: 6 - protein, 189 aa, Artificial Sequence
  Wild-type antigenic construct #2
MKAIGGSKGGSKGGSKGSWGSKGGSKGGSKGGSKGGSAVPLQSLTGEVLAHASLFVHVAGGSATSQFKGYRRRSSLNGKGESSGGSAIYEVPKEMHGNKQHLQKDFFGGSADREFQFFDHHLMESIKMGDPGGSASPLKSIWSVSTPSPIKSTLGGGSMPIGRIADCRLLWDYVYQLLSSGHHHHHHHH

SEQ ID NO: 7 - protein, 9 aa, Artificial Sequence
  ETV6 neoantigen
VLWDYVYQL

SEQ ID NO: 8 - protein, 9 aa, Artificial Sequence
  NUP210 neoantigen
AIDAALTFV

SEQ ID NO: 9 - protein, 9 aa, Artificial Sequence
  NUP210 wild-type
AIDAALTSV

SEQ ID NO: 10 - protein, 9 aa, Artificial Sequence
  ETV6 wild-type
LLWDYVYQL

(19) United States
(12) Patent Application Publication
(10) Pub. No.: US 2023/0310608 A1
(43) Pub. Date: Oct. 5, 2023

(54) IMMUNOTHERAPY

(71) Applicant: TCER ONCOLOGY AB, Skurup (SE)

(72) Inventors: Hans GRÖNLUND, Lidingö (SE); Rolf KIESSLING, Stockholm (SE); Stina WICKSTRÖM, Rönninge (SE)

(73) Assignee: TCER ONCOLOGY AB, Skurup (SE)

(21) Appl. No.: 18/003,279

(22) PCT Filed: Jul. 1, 2021
(86) PCT No.: PCT/EP2021/068267

§ 371 Date: Dec. 23, 2022

(30) Foreign Application Priority Data
Jul. 1, 2020 (GB) 2010095.4

(51) Int. Cl. A61K 39/00; A61P 35/00; C12N 5/0784; C12N 5/0783

(52) U.S. Cl. CPC A61K 39/46449; A61K 39/4611; A61K 39/00119; A61P 35/00; C12N 5/0639; C12N 5/0638; A61K 2039/5154; A61K 2039/5158; C12N 2502/1121; C12N 2502/1114

(57) ABSTRACT

The present invention provides an in vitro method for the manufacture of a dendritic cell (DC) cancer vaccine, said method comprising the steps of: (i) providing a plurality of phagocytosable particles, wherein each phagocytosable particle comprises a core and an antigenic construct tightly associated to the core, wherein the antigenic construct comprises at least one epitope peptide having an amino acid sequence corresponding to an amino acid sequence of a part of a protein or peptide known or suspected to be expressed by a cancer cell in a subject; (ii) providing a sample of DCs; and (iii) contacting the sample of DCs with the plurality of phagocytosable particles in vitro and under conditions allowing for the phagocytosis of at least one phagocytosable particle by a DC. The present invention also provides a DC cancer vaccine produced by the method of the invention, and the use a DC cancer vaccine of the invention as a medicament and for the ex vivo expansion of anticancer T-cells.

### FIELD OF THE INVENTION

The present invention relates to a dendritic cell cancer vaccine and uses thereof, especially for the treatment of cancer and its use in combination with Adoptive T-Cell Transfer for the treatment of cancer. Also disclosed herein is a method of manufacturing a dendritic cell cancer vaccine.

### BACKGROUND

There are various approaches for modulating the immune system of a subject to treat cancer; such approaches are often referred to as “immunotherapies”. Examples of immunotherapies include immune checkpoint inhibitors, adoptive cell transfer (ACT) therapies, and cancer vaccines.

There has been much success in using immune checkpoint inhibitors for treating cancer, such as the use of monoclonal antibodies that target binding interactions that are important to checkpoints of immune activation. Immune checkpoint inhibitors have been used for the treatment of various cancers, for example in the treatment of melanoma, lung cancer, bladder cancer and gastrointestinal cancers.

There has also been some success with adoptive cell transfer (ACT). For example, adoptive cell therapy (ACT) with transfer of ex vivo-expanded tumour-infiltrating lymphocytes (TILs) to lymphodepleted metastatic melanoma patients, together with high-dose IL-2 to support TIL persistence, has resulted in high rates of clinical responses (Rosenberg and Restifo, Science 2015; 348(6230): 62-8).

A significant limitation for ACT is the need to prepare sufficient quantities of anticancer T-cells, such as tumour-infiltrating lymphocytes (TILs), for administration to a subject. For example, current methods often require the use of invasive surgical procedures to remove a cancer or cancer cells of a subject in order to obtain anticancer T-cells. Furthermore, the cells obtained are few and are frequently unresponsive (anergic) due to immunosuppressive mechanisms from the cancer. This can lead to in vitro expansion of anticancer T-cells being slow, which in turn means that it can take a long time or even be impossible to obtain sufficient quantities of anticancer T-cells from TILs for therapeutic use.

Genetically engineered T-cells have been developed to overcome some of the limitations of ACT. Genetically engineered T-cells may be obtained by genetically redirecting T-cell specificity towards a patient's cancer by introduction of antigen receptors or by introducing a synthetic recognition structure, such as a chimeric antigen receptor, into a T-cell. Although genetically engineered T-cells have found success in treating hematologic cancers, the safety and selectivity of genetically engineered T-cells for treating solid cancers still requires improvement.

An alternative approach to immune checkpoint inhibitors and ACT is the administration of cancer antigens to a subject to elicit an anticancer immune response. Compositions that elicit an anticancer immune response are often referred to as “cancer vaccines”. Typically, cancer vaccines comprise a cancer antigen, such as a tumour associated antigen (TAA) or tumour specific antigen (TSA). TAAs are aberrantly expressed by a cancer cell, for example, a TAA may be a protein or peptide that is expressed by both normal cells and cancer cells, but expressed by cancer cells at a significantly higher level. TSAs are antigens that are expressed by cancer cells and not by normal cells. A variety of techniques exist for delivering cancer antigens into the body to elicit an immune response. For example, a cancer antigen may be administered as a free peptide or protein, as a nucleotide sequence encoding a cancer antigen, or presented on the surface of an antigen presenting immune cell, such as a dendritic cell (DC). Cancer vaccines have shown promise in cancer models and have been demonstrated to be safe in clinical trials. However, clinical responses have been disappointing.

Thus, despite initial promise and interest in immunotherapies for treating cancer, they have to date had limited success. As such, there remains a need for improved immunotherapies for use in the treatment of cancers.

### SUMMARY OF THE INVENTION

The present invention addresses this need. In a first aspect, the present invention provides an in vitro method for the manufacture of a dendritic cell (DC) cancer vaccine, said method comprising the steps of:
(i) providing a plurality of phagocytosable particles, wherein each phagocytosable particle comprises a core and an antigenic construct tightly associated to the core, wherein the antigenic construct comprises at least one epitope peptide having an amino acid sequence corresponding to an amino acid sequence of a part of a protein or peptide known or suspected to be expressed by a cancer cell in a subject;
(ii) providing a sample of DCs; and
(iii) contacting the sample of DCs with the plurality of phagocytosable particles in vitro and under conditions allowing for the phagocytosis of at least one phagocytosable particle by a DC.

In a further aspect, the present invention also provides a DC cancer vaccine produced by the method of the present invention. The DC cancer vaccine of the present invention finds use as a medicament. In particular, the DC cancer vaccine of the present invention finds use in the treatment or prophylaxis of a cancer in a subject.

The method of manufacturing a DC cancer vaccine of the invention provides a DC cancer vaccine that can elicit a robust anticancer immune response in a subject. As demonstrated in Example 6, the present inventors have found that DC cancer vaccines of the present invention prepared using phagocytosable particles comprising neoantigenic constructs comprising neoepitope peptides identified in HLA-A*02.01-positive melanoma tumor cells resected from a male patient with stage III auxiliary lymph node metastatic melanoma (hereinafter referred to as patient ANRU) were superior at specifically activating anticancer T-cells compared to a DC cancer vaccine prepared using soluble peptide or tumor cell lysate.

Without wishing to be bound by any particular theory, it is believed that the phagocytosable particles used in the present invention facilitate the presentation of antigenic peptides by DCs via both the MHC class I and II pathways. As such, it is expected that the DC cancer vaccine of the present invention will contain DCs that present antigenic peptides on both MHC class I and II molecules.

When antigenic peptides are presented by an MHC class II molecule, they generally activate helper T-cells (also known as CD4+ T-cells), which predominantly orchestrate immune responses by secretion of cytokines, inducing class switching of B-cells to assist the B-cells to make antibodies and stimulating activation and expansion of other T-cell types, in particular cytotoxic T-cells (e.g. CD8+ T-cells) and memory T-cells (e.g. CD8+ memory T-cells). In addition, CD4+ T-cells can directly kill other cell types (Borst et al. Nat Rev Immunol, 2018, 18(10), 635-647). Thus, administering the DC cancer vaccine of the present invention to a subject activates multiple types of immune cells, thus targeting a cancer by harnessing the whole immune system (rather than only activating CD8+ T-cell which can only attack the tumour cells directly). This is in contrast to what would be expected to occur when an antigen is provided as free peptide, tumour cell lysate, or a nucleotide construct that expresses an antigenic peptide. Such an antigen would be expected to be taken up into the cytosol of a DC, which results in antigenic peptides being presented on the cell surface solely via the MHC class I pathway. This, in turn, results in DCs that predominantly activate only CD8+ T cells. In addition to activating CD4+ and CD8+ T cells, the DC cancer vaccine of the invention leads to activation of Natural Killer (NK) cells. The NK cells are activated by soluble factors released by the DCs or released by CD4+ or CD8+ T-cells. These activated NK cells can lead to further tumour destruction, by direct killing of the cancer cells, or indirectly due to the dying tumour cells releasing tumour antigens, which may then be taken up by endogenous DCs leading to further tumour specific CD4+ or CD8+ T cells being activated through MHC class I and II presentation.

The present inventors have also found that the phagocytosable particles described herein enable the efficient manufacture of DCs suitable for use as a DC cancer vaccine. In particular, the use of the particles allows for the purification and sterilisation of the antigenic constructs described herein, thus removing contaminants, such as pathogens (e.g. bacteria, fungus and viruses), endotoxins and other antigenic contaminants from the antigenic constructs before internalisation and presentation of a DC, and without diminishing the effectiveness of the resulting DCs and their ability to elicit an anticancer T-cell response. This is particularly advantageous because the removal of contaminants from the antigenic constructs reduces non-specific contamination of the DCs and thus reduces non-specific immune-responses in the subject after administration of the DC cancer vaccine.

In a further aspect, the present invention also provides a method of treating or preventing cancer comprising a step of administering to a subject in need thereof the DC cancer vaccine of the present invention. Also provided is the use of a DC cancer vaccine of the present invention for the manufacture of a medicament for treatment or prophylaxis of cancer.

Preferably, the method of treating or preventing cancer further comprises the steps of:
a) harvesting anticancer T-cells from a blood sample from the subject;
b) expanding the anticancer T-cells in vitro; and
c) administering a therapeutic dose of the expanded anticancer T-cells to the subject;

wherein steps a), b) and c) are performed before administering the DC cancer vaccine to the subject, and/or wherein steps a), b) and c) are performed after administering a DC cancer vaccine of the invention to the subject. Thus, in a further aspect, the present invention also provides a method for the in vitro expansion of anticancer T-cells, said method comprising the steps of:
ba) providing a phagocytosable particle comprising a core and an antigenic construct tightly associated to the core, wherein the antigenic construct comprises at least one epitope having an amino acid sequence corresponding to an amino acid sequence of a part of a protein or peptide known or suspected to be expressed by a cancer cell in a subject;
bb) providing an antigen-presenting cell (APC);
bc) contacting the phagocytosable particle with the APC from step bb) in vitro, and under conditions allowing phagocytosis of the phagocytosable particle by the APC;
bd) providing an anticancer T-cell sample harvested from a blood sample from the subject; and
be) contacting the anticancer T-cell sample with the APC from step bb) in vitro, and under conditions allowing specific activation and expansion of anticancer T-cells in response to antigen presented by the APC;

wherein the subject is one whom has not previously been administered a DC cancer vaccine of the invention, or wherein the subject is one whom has previously been administered a DC cancer vaccine of the invention. In embodiments wherein steps a), b) and c) are performed before administering a DC cancer vaccine of the invention to the subject, the administration of a DC cancer vaccine after administration of a therapeutic dose of expanded anticancer T-cells in step c) acts to further stimulate the expansion of anticancer T-cells in the subject therefore prolonging the anticancer effects of the therapeutic dose of expanded anticancer T-cells administered to the subject in step c). In embodiments wherein steps a), b) and c) are performed after administering the DC cancer vaccine of the invention to the subject, the prior administration of the DC cancer vaccine of the invention increases the level of circulating anticancer T-cells in the subject, thus providing a readily accessible source of anticancer T-cells that can be harvested from the blood of the subject. Such anticancer T-cells can then be activated and expanded in vitro and administered to the subject as an adoptive cell transfer therapy.

### BRIEF DESCRIPTION OF THE FIGURES

FIG. 1 shows the effect of phagocytosable particle size on T-cell activation. A proliferation assay (with thymidine incorporation) was used to assess the number of splenocytes obtained from ovalbumin sensitized mice. Comparison of ovalbumin coupled to differently sized phagocytosable particle with a diameter of 5.6 μm, 1 μm and 0.2 μm are shown. P-values determined using students T-test and written indicated when p<0.05 found. Staples denote SD.

FIG. 2A shows the confocal microscope images of PBMCs with intracellular, phagocytosed particles. Three sizes of phagocytosable particle are shown (4.5 μm, 2.8 μm or 1 μm) following incubation for 18 h at 37° C. of PBMCs with the phagocytosed particles.

FIG. 2B shows the cellular uptake of phagocytosable particles of two sizes (4.5 μm or 2.8 μm) after incubation with PBMCs for 18 h at 37° C., as assessed by manual counting.

FIG. 2C shows the cellular uptake of phagocytosable particles of three sizes (4.5 μm, 2.8 μm or 1 μm) after incubation in PBMCs for 18 h at 37° C., as assessed by volume calculation (*p<0.05 **p<0.01 ***p<0.001, calculated using Student's T-test).

FIG. 3A shows the relative increase in the level of IFNγ-production in PBMCs from CMV-sensitive healthy donors (n=2) stimulated with phagocytosable particles of three sizes (4.5 μm, 2.8 μm or 1 μm) compared to non-stimulated cells, as assessed in the FluoroSpot assay of Example 4(iv).

FIG. 3B shows the relative increase in the level of IL22-production in PBMCs from a CMV-sensitive healthy donor (n=1) stimulated with phagocytosable particles of three sizes (4.5 μm, 2.8 μm or 1 μm) compared to non-stimulated cells, as assessed in the FluoroSpot assay of Example 4(iv).

FIG. 3C shows the relative increase in the level of IL17-production in PBMCs from CMV-sensitive healthy donor (n=1) stimulated with phagocytosable particles of three sizes (4.5 μm, 2.8 μm or 1 μm) compared to non-stimulated cells, as assessed in the FluoroSpot assay of Example 4(iv).

FIG. 3D shows the relative increase in the dual-cytokine production of IFNγ and IL17 in PBMCs from a CMV-sensitive healthy donor (n=1) stimulated with phagocytosable particles of three sizes (4.5 μm, 2.8 μm or 1 μm) compared to non-stimulated cells, as assessed in the FluoroSpot assay of Example 4(iv).

FIG. 3E shows the relative increase in the dual-cytokine production of IL22 and IL17 in PBMCs from a CMV-sensitive healthy donor (n=1) stimulated with phagocytosable particles of three sizes (4.5 μm, 2.8 μm or 1 μm) compared to non-stimulated cells, as assessed in the FluoroSpot assay of Example 4(iv).

FIGS. 4 and 5 show the percentage of CD8+ cells that express CD107 in a T-cell sample co-cultured with a DC vaccine and then exposed to ANLU tumor cells for 6 hours. The data shown in FIG. 4 relate to CD8+ T-cells derived from healthy donor 1, and the data shown in FIG. 5 relate to CD8+ T-cells derived from healthy donor 2.

FIG. 6 shows the percentage of CD8+ cells that express CD107 (FIG. 6A) or IFNγ (FIG. 6B) in a T-cell sample co-cultured with a DC vaccine and then exposed to ANLU tumor cells for 6 hours or 24 hours. The data shown in FIG. 6 relate to CD8+ T-cells derived from healthy donor 3.

FIG. 7 shows the percentage of CD8+ cells that express CD107 in a T-cell sample co-cultured with a DC vaccine and then exposed to ANLU tumor cells for 6 hours. The data shown in FIG. 7 relate to CD8+ T-cells derived from healthy donor 4.

FIG. 8 shows the percentage of CD8+ cells that express CD107 in a T-cell sample co-cultured with a DC vaccine and then exposed to ANLU tumor cells for 6 hours. The data shown in FIG. 8 relate to CD8+ T-cells derived from Patient ANRU.

FIG. 9 shows the construct design of neoantigenic construct #1 (FIG. 9A, SEQ ID NO: 1) and neoantigenic construct #2 (FIG. 9B, SEQ ID NO: 2). Neoantigenic construct #1 comprises six neoepitope peptide sequences referred to as PLCL2, CDCA2, CAPN3, ATP8B4, NUP210 and ETV6, and neoantigenic construct #2 comprises the six neoepitope peptide sequences referred to as PLCL2, CDCA2, CAPN3, ATP8B4, ANK3 and ETV6. Each neoepitope peptide sequence is separated by a GGS linker. The neoepitope peptide sequences used in neoantigenic constructs #1 and #2 were identified in tumor cells resected from patient ANRU. The construct designs shown in FIGS. 9A and 9B were used to prepare neoantigenic construct #1 (SEQ ID NO: 3) and #2 (SEQ ID NO: 4), which additionally comprise an N-terminal initiation sequence and polylysine tag, and a C-terminal GSS linker and polyhistidine tag.

### DETAILED DESCRIPTION OF THE INVENTION

The Antigenic Constructs and Epitope Peptides

A phagocytosable particle for use in the present invention comprises an antigenic construct tightly associated to a core. An antigenic construct described herein comprises at least one epitope peptide having an amino acid sequence corresponding to an amino acid sequence of a part of a protein or peptide known or suspected to be expressed by a cancer cell in a subject. Preferably the subject is a mammal and more preferably, a human. The cancer cell in the subject may be a cell from any form of cancer, for example a solid cancer, a metastatic solid cancer or a hematologic malignancy. Types of solid cancer include sarcomas, carcinomas, and lymphomas. Types of hematologic cancer include leukaemia, lymphomas, myelomas and myelodysplastic syndromes (lymphomas may be classed as both a solid cancer and a hematologic malignancies). In certain embodiments, the cancer cell in the subject is a skin cancer cell, breast cancer cell, colon cancer cell, liver cancer cell, lung cancer cell, pancreatic cancer cell, prostate cancer cell, ovarian cancer cell, bladder cancer cell, cervical cancer cell, sarcoma cell, head-and-neck cancer cell or renal cancer cell.

The epitope peptide for use in the invention may have an amino acid sequence corresponding to an amino acid sequence of a tumour associated antigen (TAA). Examples of TAAs include MAGE-A3, gp100, MART-1, NUP210, ETV6, CEA, PSA, p53, NYESO-1. Alternatively, the epitope peptide may have an amino acid sequence corresponding to an amino acid sequence of a tumour specific antigen (TSA).

An epitope peptide of the invention has an amino acid sequence that is 3 to 200 amino acids in length (for example 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, 125, 150, 175 or 200 amino acids in length). Preferably the epitope peptide has an amino acid sequence that is 3 to 50 amino acids in length (for example 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50 amino acids in length), more preferably 3 to 30 amino acids in length (for example 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length), more preferably 3 to 25 amino acids (for example 3, 4, 5, 6, 7, 8, 9, 10, 11, 12, 13, 14, 15, 16, 17, 18, 19, 20, 21, 22, 23, 24, or 25 amino acids in length), more preferably 5 to 25 amino acids (for example 5, 6, 7, 8, 9, 10, 11, 12, 13, 14, 15, 16, 17, 18, 19, 20, 21, 22, 23, 24, or 25 amino acids in length), more preferably 8 to 25 amino acids (for example 8, 9, 10, 11, 12, 13, 14, 15, 16, 17, 18, 19, 20, 21, 22, 23, 24, or 25 amino acids in length), and even more preferably 11 to 25 amino acids in length (for example 11, 12, 13, 14, 15, 16, 17, 18, 19, 20, 21, 22, 23, 24, or 25 amino acids in length). In one preferred embodiment, the epitope peptide is 3 to 25 amino acids, 5 to 25 amino acids, 10 to 25 amino acids, 11 to 25 amino acids, 12 to 25 amino acids, 13 to 25 amino acids, 15 to 25 amino acids, 17 to 25 amino acids, 19 to 25 amino acids, 20 to 25 amino acids in length, or 21 to 25 amino acids in length. In another preferred embodiment, the epitope peptide is 3 to 23 amino acids, 5 to 23 amino acids, 10 to 23 amino acids, 11 to 23 amino acids, 12 to 23 amino acids, 13 to 23 amino acids, 15 to 23 amino acids, 17 to 23 amino acids, 19 to 23 amino acids, 20 to 23 amino acids, or 21 to 23 amino acids in length. In another preferred embodiment, the epitope peptide is 3 to 21 amino acids, 5 to 21 amino acids, 10 to 21 amino acids, 11 to 21 amino acids, 12 to 21 amino acids, 13 to 21 amino acids, 15 to 21 amino acids, 17 to 21 amino acids, or 19 to 21 amino acids in length. In another preferred embodiment, the epitope peptide is 3 to 19 amino acids, 5 to 19 amino acids, 10 to 19 amino acids, 11 to 19 amino acids, 12 to 21 amino acids, 13 to 21 amino acids, 15 to 21 amino acids, or 17 to 19 amino acids in length. In another preferred embodiment, the epitope peptide is 3 to 17 amino acids, 5 to 17 amino acids 10 to 17 amino acids, 11 to 17 amino acids, 12 to 17 amino acids, 13 to 17 amino acids or 15 to 17 amino acids in length. In another preferred embodiment, the epitope peptide is 3 to 15 amino acids, 3 to 15 amino acids 10 to 15 amino acids, 11 to 15 amino acids, 12 to 15 amino acids or 13 to 15 amino acids in length. In another preferred embodiment, the epitope peptide is 3 to 19 amino acids, 5 to 17 amino acids, 5 to 15 amino acids, 5 to 13 amino acids, or 5 to 10 amino acids in length. In another preferred embodiment, the epitope peptide may be 3 to 19 amino acids, 5 to 17 amino acids, 3 to 15 amino acids, 3 to 10 amino acids, or 5 to 10 amino acids in length. In another preferred embodiment, the epitope peptide may be 3 to 19 amino acids, 3 to 17 amino acids, 3 to 13 amino acids, 3 to 10 amino acids, or 3 to 7 amino acids in length.

The antigenic construct may comprise one epitope peptide, or it may comprise more than one epitope peptide. For example, the antigenic construct may comprise 1 to 50 epitope peptides (e.g. 1, 2, 3, 4, 5, 6, 7, 8, 9, 10, 11, 12, 13, 14, 15, 16, 17, 18, 19, 20, 21, 22, 23, 24, 25, 23, 24, 25, 26, 27, 28, 29, 30, 31, 32, 33, 34, 35, 36, 37, 38, 39, 40, 41, 42, 43, 44, 45, 46, 47, 48, 49, 50 epitope peptides). Preferably, the antigenic construct comprises 1 to 20 epitope peptides (e.g. 1, 2, 3, 4, 5, 6, 7, 8, 9, 10, 11, 12, 13, 14, 15, 16, 17, 18, 19 or 20 epitope peptides), more preferably, 1 to 15 epitope peptides (e.g. 1, 2, 3, 4, 5, 6, 7, 8, 9, 10, 11, 12, 13, 14 or 15 epitope peptides), more preferably 1 to 10 epitope peptides (e.g. 1, 2, 3, 4, 5, 6, 7, 8, 9 or 10 epitope peptides), more preferably 1 to 8 epitope peptides (e.g. 1, 2, 3, 4, 5, 6, 7 or 8 epitope peptides), more preferably 1 to 6 epitope peptides (e.g. 1, 2, 3, 4, 5 or 6 epitope peptides), and even more preferably 1 to 5 epitope peptides (e.g. 1, 2, 3, 4, or 5 epitope peptides). It is especially preferred that the antigenic construct comprises 1 to 5 epitope peptides (e.g. 1, 2, 3, or 4 epitope peptides), and even more preferably 3 to 5 epitope peptides, for example 3, 4 or 5 epitope peptides.

In certain embodiments, the antigenic construct comprises two or more epitope peptides. For example, the antigenic construct may comprise 2 to 50 epitope peptides (e.g. 2, 3, 4, 5, 6, 7, 8, 9, 10, 11, 12, 13, 14, 15, 16, 17, 18, 19, 20, 21, 22, 23, 24, 25, 23, 24, 25, 26, 27, 28, 29, 30, 31, 32, 33, 34, 35, 36, 37, 38, 39, 40, 41, 42, 43, 44, 45, 46, 47, 48, 49, 50 epitope peptides). Preferably, the antigenic construct comprises 2 to 20 epitope peptides (e.g. 2, 3, 4, 5, 6, 7, 8, 9, 10, 11, 12, 13, 14, 15, 16, 17, 18, 19 or 20 epitope peptides), more preferably, the antigenic construct comprises 2 to 15 epitope peptides (e.g. 2, 3, 4, 5, 6, 7, 8, 9, 10, 11, 12, 13, 14 or 15, epitope peptides), more preferably 2 to 10 epitope peptides (e.g. 2, 3, 4, 5, 6, 7, 8, 9 or 10 epitope peptides), more preferably 2 to 8 epitope peptides (e.g. 2, 3, 4, 5, 6, 7 or 8 epitope peptides), more preferably 2 to 6 epitope peptides (e.g. 2, 3, 4, 5 or 6), and even more preferably 2 to 5 epitope peptides (e.g. 2, 3, 4, or 5 epitope peptides). It is especially preferred that the antigenic construct comprises 2 to 5 epitope peptides (e.g. 2, 3, 4, or 5 epitope peptides), and even more preferably 3 to 5 epitope peptides, for example 3, 4 or 5 epitope peptides.

In one preferred embodiment, the antigenic construct comprises three or more epitope peptides. For example, the antigenic construct may comprise 3 to 50 epitope peptides (e.g. 3, 4, 5, 6, 7, 8, 9, 10, 11, 12, 13, 14, 15, 16, 17, 18, 19, 20, 21, 22, 23, 24, 25, 23, 24, 25, 26, 27, 28, 29, 30, 31, 32, 33, 34, 35, 36, 37, 38, 39, 40, 41, 42, 43, 44, 45, 46, 47, 48, 49, 50 epitope peptides). Preferably, the antigenic construct may comprise 3 to 20 epitope peptides (e.g. 3, 4, 5, 6, 7, 8, 9, 10, 11, 12, 13, 14, 15, 16, 17, 18, 19 or 20 epitope peptides), more preferably, the antigenic construct may comprise 3 to 15 epitope peptides (e.g. 3, 4, 5, 6, 7, 8, 9, 10, 11, 12, 13, 14 or 15 epitope peptides), more preferably 3 to 10 epitope peptides (e.g. 3, 4, 5, 6, 7, 8, 9 or 10 epitope peptides), more preferably 3 to 8 epitope peptides (e.g. 3, 4, 5, 6, 7 or 8 epitope peptides), more preferably 3 to 6 epitope peptides (e.g. 3, 4, 5 or 6), and even more preferably 3 to 5 epitope peptides (e.g. 3, 4, or 5 epitope peptides). It is especially preferred that the antigenic construct comprises 3 to 5 epitope peptides (e.g. 3, 4 or 5 epitope peptides), and even more preferably 5 epitope peptides.

In another preferred embodiment, the antigenic construct comprises four or more epitope peptides (e.g. 4 epitope peptides), five or more epitope peptides (e.g. 5 epitope peptides), or six or more epitope peptides (e.g. 6 epitope peptides).

For the avoidance of doubt, in embodiments where the antigenic construct may comprise more than one epitope peptide (for example, one or more epitope peptides, two or more epitope peptides, or three or more epitope peptides), each epitope peptide is an epitope peptide as described herein for use in the present invention (i.e. each epitope peptide of a antigenic construct has an amino acid sequence corresponding to an amino acid sequence of a part of a protein or peptide known or suspected to be expressed by a cancer cell in the subject). In such embodiments, each epitope peptide may independently have any of the properties and/or characteristics of an epitope peptide described herein.

In embodiments where the antigenic construct may comprise more than one epitope peptide (e.g. one or more epitope peptides, two or more epitope peptides or three or more epitope peptides), each epitope peptide may have the same amino acid sequence, or the epitope peptides may have different amino acid sequences (i.e. some epitope peptides of a antigenic construct may have different amino acid sequences, or all of the epitope peptides of an antigenic construct may have different amino acid sequences).

In embodiments where the antigenic construct comprises more than one epitope peptide, some or all of the epitope peptides have different amino acid sequences. Preferably, all of the epitope peptides have different amino acid sequences.

In embodiments where the antigenic construct comprises more than one epitope peptide, each epitope peptide has the same amino acid sequence.

In embodiments where the antigenic construct may comprise more than one epitope peptide (e.g. one or more epitope peptides, two or more epitope peptides or three or more epitope peptides), and some of the epitope peptides have different amino acid sequences, or all of the epitope peptides have different amino acid sequences, the amino acid sequences may be different because:
the protein or peptide known or suspected to be expressed by a cancer cell in the subject are different; or
the protein or peptide known or suspected to be expressed by a cancer cell in the subject is the same, but the part of the protein or peptide known or suspected to be expressed by a cancer cell in the subject that the amino acid sequence of the epitope peptide corresponds to are different.

In preferred embodiments, the amino acid sequences are different because for each epitope peptide the protein or peptide known or suspected to be expressed by a cancer cell in the subject is different.

In embodiments where an antigenic construct comprises more than one epitope peptide (e.g. two or more epitope peptides or three or more epitope peptides), the epitope peptides may be directly linked, or linked via a spacer moiety.

In preferred embodiments where an antigenic construct comprises more than one epitope peptide (e.g. one or more epitope peptides, two or more epitope peptides or three or more epitope peptides), the epitope peptides are covalently linked.

An antigenic construct comprising more than one epitope peptide (e.g. two or more epitope peptides or three or more epitope peptides), may comprise epitope peptides that are directly linked and/or epitopes that are linked via a spacer moiety. If more than one spacer moiety is present in a neoantigenic construct, the spacer moieties of an antigenic construct may all be the same or they may be different.

In embodiments where an antigenic construct comprises more than one epitope peptide (e.g. one or more epitope peptides, two or more epitope peptides or three or more epitope peptides), the epitope peptides are each linked via a spacer moiety.

A spacer moiety may be a short sequence of amino acids, for example 1 to 15 amino acids, preferably 1 to 10 amino acids, and more preferably 1 to 5 amino acids (for example, 1, 2, 3, 4 or 5 amino acids). The spacer moiety may comprise a random combination of amino acids; or a synthetic amino acid sequence, such as a polylysine, polyarginine, polyglycine, polyalanine or polyhistidine amino acid sequence. Preferably, the spacer moiety comprises the motif VVR and/or GGS.

The structure of the antigenic construct comprising two linked epitope peptides may be as follows:
-[1^{st} epitope peptide]-[spacer moiety]-[2^{st} epitope peptide].

The structure of the antigenic construct comprising three linked epitopes may be as follows:
-[1^{st} epitope peptide]-[spacer moiety]-[2^{nd} epitope peptide]-[spacer moiety]-[3^{rd} epitope peptide].

The structure of the neoantigenic construct comprising four linked epitopes may be as follows:
-[1^{st} epitope peptide]-[spacer moiety]-[2^{nd} epitope peptide]-[spacer moiety]-[3^{rd} epitope peptide]-[spacer moiety]-[4^{th} epitope peptide].

The structure of the antigenic construct comprising five linked epitopes may be as follows:
-[1^{st} epitope peptide]-[spacer moiety]-[2^{nd} epitope peptide]-[spacer moiety]-[3^{rd} epitope peptide]-[spacer moiety]-[4^{th} epitope peptide]-[spacer moiety]-[5^{th} epitope peptide].

The structure of the antigenic construct comprising n linked epitope peptide may be as follows:
-[1^{st} epitope peptide]-[spacer moiety]-[2^{nd} epitope peptide]-[spacer moiety]-[3^{rd} epitope peptide]-[spacer moiety]-[n^{th} epitope peptide].

When the antigenic construct comprises one or more epitope peptide, the antigenic construct may consist of the one or more epitope peptide, and optionally any spacer moiety(s). Alternatively, when the antigenic construct comprises one or more epitope peptide, and optionally any spacer moiety(s) and a further amino acid sequence. Such a further amino acid sequence may, for example, be a random combination of amino acids (in particular random sequences having a high proportion of histidine and/or cysteine residues); or a synthetic amino acid sequence, such as a polylysine, polyarginine, polyglycine, polyalanine, polyhistidine or polycysteine amino acid sequence (and preferably a polyhistidine or polycysteine). Such a further amino acid sequence may be used, for example, as a linker and/or a spacer between the core of the phagocytosable particle and an epitope peptide of antigenic construct tightly associated to it, or used to tightly associate the antigenic construct to the phagocytosable particle. For example, metal chelates can bind proteins and peptides containing histidine or cysteine with great strength. Thus, cores with metal chelates can non-covalently bind to an antigenic construct comprising a polyhistidine or polycysteine synthetic amino acid sequence and/or a random combination of amino acids having a high proportion of histidine and/or cysteine residues as a further amino acid sequence.

The length of an antigenic construct for use according to the invention depends on, for example, the number of epitope peptides in the antigenic construct, and the length of each epitope peptide in the antigenic construct, as well as the length of any spacer moieties that may be present if there is more than one epitope peptide, and the length of any further amino acids sequences that may be present. In certain preferred embodiments, the antigenic construct may have an amino acid sequence that is 3 to 300 amino acids, and preferably 10 to 250, more preferably 10 to 200 and more preferably 10 to 180 amino acids in length. For example, the antigenic construct may comprise 11 to 150 amino acids, 11 to 140 amino acids, 33 to 120 amino acids, 42 to 140 amino acids, 11 to 112 amino acids, 33 to 112 amino acids 2, 42 to 112 amino acids, 11 to 100 amino acids, 33 to 100 amino acids, 42 to 100 amino acids, 11 to 84 amino acids, 33 to 84 amino acids, 42 to 84 amino acids, 11 to 60 amino acids, 33 to 60 amino acids, or 42 to 60 amino acids, 11 to 50 amino acids, 33 to 50 amino acids, or 42 to 50 amino acids in length in length.

Antigenic constructs for use according to the invention can be prepared recombinantly (for example, in E. coli, mammalian cells or insect cells), synthetically (for example, using standard organic chemistry techniques, such as solution or solid phase peptide synthesis), or they may be prepared from polypeptides isolated from a native protein or peptide derived from an animal source, for example a human source. Preferably, antigenic constructs for use in the present invention are prepared recombinantly (for example, in E. coli, mammalian cells or insect cells). More preferably, antigenic constructs for use in the present invention are prepared recombinantly in E. coli.

Neoepitopes

In preferred embodiments, the epitope peptide has an amino acid sequence corresponding to an amino acid sequence of a tumour specific antigen (TSA). A particular example of a TSA is a neoantigen. It is preferred that the epitope peptide is a neoepitope peptide. An antigenic construct comprising at least one neoepitope peptide may be referred to herein as a neoantigenic construct.

A neoepitope peptide is a peptide having an amino acid sequence corresponding to an amino acid sequence of a part of a protein or peptide known or suspected to be expressed by a cancer cell in the subject, wherein the part of the protein or peptide has at least one somatic mutated amino acid. The at least one mutated somatic amino acid may have a direct influence on tumour progression (i.e. a driver mutation), or may have no direct or indirect influence on tumour progression (i.e. a passenger mutation). It should be understood that the present invention allows for the use of driver and passenger mutated somatic amino acids.

A “somatic mutated amino acid” of a neoepitope peptide is an amino acid that is different or not present in the part of the protein or peptide corresponding to the neoepitope peptide amino acid sequence when that part of the protein or peptide is expressed by a non-cancerous cell (e.g. a somatic cell). For example, a “somatic mutated amino acid” of a neoepitope peptide may be a deletion (i.e. an amino acid that has been deleted), an addition (i.e. an amino acid that has been added) or a substitution (i.e. an amino acid that has been substituted for a different amino acid). Such somatic mutated amino acids may also be referred to as a “cancer-specific somatic mutated amino acid” because the somatic mutated amino acids are present in a cancer cell, but not in a normal cell (e.g. a somatic cell). Preferably, the somatic mutated amino acid(s) of a neoepitope peptide is/are a substitution (i.e. one or more amino acid has been substituted for a different amino acid).

Mutated somatic amino acids in a protein or peptide expressed by a cell can occur as a result of infidelity of DNA replication occurring at each cell division creating substitutions, deletions or insertions of nucleotides into the DNA of a cell. Nucleotide substitutions can result in a different amino acid being coded for compared to the amino acid coded for by the somatic non-mutated nucleic acid sequence, thus resulting in a different amino acid in the protein/peptide compared to the protein/peptide in a non-cancerous cell (e.g. a somatic cell). Nucleotide insertion(s) and/or deletion(s) can result in a reading frame error (i.e. a “frameshift mutation”), thus resulting in a new amino acid sequence at the protein level (i.e. nucleotide insertion(s) or deletion(s) altering the reading frame of the DNA and thus altering most or all of the amino acids encoded by the DNA after the mutation compared to a non-cancerous cell (e.g. somatic cell)). Additionally, or alternatively, an insertion and/or deletion can result in the introduction of a stop codon, thus resulting in a truncated protein at the protein level. A nucleotide substitution can individually alter codon(s) and result in amino acid substitution(s) at the protein level and/or the introduction of a stop codon, thus resulting in a truncated protein at the protein level. Additionally, or alternatively, an insertion(s) and/or deletion(s) can result for erroneous RNA splicing.

Neoepitope peptides for use in the present invention are peptides having an amino acid sequence corresponding to an amino acid sequence of a part of a protein or peptide known or suspected to be expressed by a cancer cell in the subject, wherein the part of the protein or peptide has at least one somatic mutated amino acid (e.g. 1, 2, 3, 4, or 5, or more, somatic mutated amino acids).

A mutated protein or peptide known or suspected to be expressed by a cancer cell in a subject may also be referred to as a “cancer-specific mutated protein or peptide”. That is because the mutated protein or peptide is known or suspected to be expressed in a cancer cell, but not a non-cancerous cell (e.g. a somatic cell).

A cancer-specific mutated protein or peptide, and its amino acid sequence that a neoepitope peptide amino acid sequence can correspond to, may be identified using a variety of techniques. For example, a cancer-specific mutated protein or peptide, and its amino acid sequence including its cancer-specific somatic mutated amino acid(s), may be identified from publicly available protein databases, such as the COSMIC database (Forbes et al., Nucleic Acids Res, 45(D1), D777-D783, the database is accessible at http://cancer.sanger.ac.uk/cosmic). Somatic mutated amino acid sequences identified from the COSMIC database, or similar databases, are referred to herein as “predicted neoepitope peptides”. Neoantigenic constructs consisting of one or more “predicted neoepitope peptides” are referred to herein as a “predicted neoantigenic constructs”.

In an alternative or additional approach to identify a cancer-specific mutated protein or peptide, and its amino acid sequence that a neoepitope peptide amino acid sequence can correspond to, the genome, exome transcriptome and/or proteome of a cancer cell obtained from a cancer in a subject may be established, and thus the mutations in a cancer cell deduced. That can be done, for example, by comparison of the proteome, genome, exome or transcriptome derived data with reference nucleotide sequences or amino acid sequence. Suitable reference sequences may be obtained from the genome, exome or transcriptome of a non-cancerous cell (e.g. a somatic cell) obtained from the subject or from publicly available nucleotide or protein databases, such as the UniProt databases (https://www.uniprot.org/) and the EBI expression atlas (https://www.ebi.ac.uk/gxa/home), which provides information on proteins and peptides which are expressed in tissues and cancer cell lines. In an alternative or additional approach, a cancer-specific mutated protein or peptide, and its amino acid sequence that a neoepitope peptide amino acid sequence can correspond to, may be one that has been previously identified by analysis of the genome, exome, transcriptome and/or proteome of a tumour of a subject. Suitable techniques for sequencing the genome, exome or transcriptome of a cancer cell or a normal cell are known in the art, and include, for example Sanger sequencing and next-generation sequencing. Suitable techniques for obtaining proteome data include Multiple Reaction Monitoring (MRM) mass spectrometry. Determination of cancer-specific mutations at the proteome level is particularly useful for identifying mutations that cannot be predicted from DNA and/or RNA sequences, for example in instances where splice sites are erroneously missed leading to translation of intragenic regions of a gene.

Somatic mutated amino acid sequences identified from genome, exome, transcriptome or proteome data obtained from a subject are referred to herein as “personalised neoepitope peptides”. Neoantigenic constructs consisting of one or more “personalised neoepitope peptides” are referred to herein as a “personalised neoantigenic constructs”.

A neoepitope peptide for use in the present invention has one or more somatic mutated amino acids. For example, it may have one somatic mutated amino acid, or more than one somatic mutated amino acids, i.e. from two to all of the amino acids in the part of the protein or peptide may be mutated. For example, a neoepitope peptide for use in the invention may have 1 to 10 (for example 1, 2, 3, 4, 5, 6, 7, 8, 9 or 10) somatic mutated amino acids, more preferably 1 to 8 (for example 1, 2, 3, 4, 5, 6, 7 or 8) mutated amino acids; or, for example, 1 to 6 (for example 1, 2, 3, 4, 5 or 6) mutated amino acids; or, for example, 1 to 5 (for example 1, 2, 3, 4 or 5) somatic mutated amino acids; or, for example, 1 to 4 (for example 1, 2, 3, or 4) somatic mutated amino acids. In preferred embodiments, the neoepitope peptide of the invention has 1, 2 or 3 somatic mutated amino acids. In preferred embodiments, the neoepitope peptide has 1 or 2 somatic mutated amino acids. Even more preferably, the neoepitope peptide has one somatic mutated amino acid.

In preferred embodiments, the neoepitope peptide is 10 to 25 amino acids, 10 to 23 amino acids, 10 to 21 amino acids, 10 to 19 amino acids, 10 to 17 amino acids, 10 to 15 amino acids and comprises 1, 2, 3, 4, or 5, or all, somatic mutated amino acids. Preferably, the neoepitope peptide is 10 to 25 amino acids, 10 to 23 amino acids, 10 to 21 amino acids, 10 to 19 amino acids, 10 to 17 amino acids, 10 to 15 amino acids in length and comprises 1, 2 or 3 somatic, or all, mutated amino acids. More preferably, the neoepitope peptide is 10 to 25 amino acids, 10 to 23 amino acids, 10 to 21 amino acids, 10 to 19 amino acids, 10 to 17 amino acids, 10 to 15 amino acids in length and the neoepitope comprises 1 or 2 somatic, or all, mutated amino acids. Even more preferably, the neoepitope peptide is 10 to 25 amino acids, 10 to 23 amino acids, 10 to 21 amino acids, 10 to 19 amino acids, 10 to 17 amino acids, 10 to 15 amino acids in length and comprises one somatic, or all, mutated amino acid. Even more preferably, the neoepitope peptide is 10 to 25 amino acids, 10 to 23 amino acids, 10 to 21 amino acids, 10 to 19 amino acids, 10 to 17 amino acids, 10 to 15 amino acids in length and comprises one somatic mutated amino acid.

In other preferred embodiments, the neoepitope peptide is 3 to 25 amino acids, 3 to 17 amino acids, 3 to 15 amino acids, 3 to 10 amino acids, or 5 to 10 amino acids in length, and comprises 1, 2, 3, 4, or 5, or all, somatic mutated amino acids. Preferably, the neoepitope peptide is 3 to 25 amino acids, 3 to 17 amino acids, 3 to 15 amino acids, 3 to 10 amino acids, or 5 to 10 amino acids in length, and comprises 1, 2 or 3, or all, somatic mutated amino acids. More preferably, the neoepitope peptide is 3 to 25 amino acids, 3 to 17 amino acids, 3 to 15 amino acids, 3 to 10 amino acids, or 5 to 10 amino acids in length, and comprises 1 or 2, or all, somatic mutated amino acids. Even more preferably, the neoepitope peptide is 3 to 25 amino acids, 3 to 17 amino acids, 3 to 15 amino acids, 3 to 10 amino acids, or 5 to 10 amino acids in length, and comprises one, or all, somatic mutated amino acid. Even more preferably, the neoepitope peptide is 3 to 25 amino acids, 3 to 17 amino acids, 3 to 15 amino acids, 3 to 10 amino acids, or 5 to 10 amino acids in length, and comprises one, or all, somatic mutated amino acid.

In embodiments, a neoepitope peptide has most or all somatic mutated amino acids. Even more preferably, a neoepitope peptide has all somatic mutated amino acids. Such neoepitope peptides having most or all somatic mutated amino acids may correspond to a part of a protein or a peptide resulting from a frameshift type mutation in the DNA of a cell.

The one or more amino acid mutations of a neoepitope peptide for use in the present invention may be located at any amino acid position within the neoepitope peptide amino acid sequence. In preferred embodiments, at least one of the somatic mutated amino acids (or the one somatic mutated amino acid in embodiments where there is only one somatic mutated amino acid in the neoepitope peptide) is located in the central portion of the neoepitope peptide. For example, when a neoepitope peptide amino acid sequence is at least 3 amino acids in length (for example at least 5 amino acids in length or at least 7 amino acids in length), the central portion of the neoepitope peptide is the central 1 amino acid of the sequence when the neoepitope peptide has an odd number of amino acids in its sequence; or the central 2 amino acids when the neoepitope peptide has an even number of amino acids in its sequence. For example, when a neoepitope peptide amino acid sequence is at least 9 amino acids in length, the central portion of the neoepitope peptide is the central 3 amino acids of the sequence (and preferably the 1 central amino acid) when the neoepitope peptide has an odd number of amino acids in its sequence; or the central 4 amino acids (and preferably the 2 central amino acid) when the neoepitope peptide has an even number of amino acids in its sequence. For example, when a neoepitope peptide amino acid sequence is at least 11 amino acids in length, the central portion of the neoepitope peptide is the central 5 amino acids of the sequence (and preferably the 1 central amino acid) when the neoepitope peptide has an odd number of amino acids in its sequence; or the central 6 amino acids when the neoepitope peptide has an even number of amino acids in its sequence. More preferably, when a neoepitope peptide amino acid sequence is at least 11 amino acids in length, the central portion of the neoepitope peptide is the central 3 amino acids (and preferably the 1 central amino acid) of the sequence when the neoepitope peptide has an odd number of amino acids in its sequence; or the central 4 amino acids (and preferably the 2 central amino acid) when the neoepitope peptide has an even number of amino acids in its sequence.

In embodiments, at least one of the somatic mutated amino acids (or the one somatic mutated amino acid in embodiments where there is only one somatic mutated amino acid in the neoepitope peptide) of a neoepitope peptide is located at the central position of the neoepitope peptide when the neoepitope peptide has an odd number of amino acids in its sequence, or at either of the two most central positions of the amino acid sequence when the neoepitope peptide has an even number of amino acids in its sequence.

In embodiments, most or all of the amino acids of the neoepitope peptide are somatic mutated amino acids. In such embodiments the somatic mutated amino acids in the protein or peptide expressed by a cancer cell may have occurred due to an error in the reading frame of the encoding DNA (i.e. due to a frameshift mutation) resulting in all or most of the amino acids in the part of the protein or peptide being different to the part of the protein or peptide expressed in a normal non-cancerous cell.

Neoepitope peptides having an amino acid sequence that corresponds to an amino acid sequence of a part of a cancer-specific protein or peptide that is 3 to 25 amino acids in length, and comprising one or more somatic mutated amino acids, are particularly effective at eliciting an anticancer immune response in a subject when displayed on the surface of DCs in the DC cancer vaccine of the invention. The use of such neoepitope peptides also reduces the risk of eliciting an autoimmune or non-cancer specific immune response in the subject.

In embodiments where the neoantigenic construct may comprise more than one neoepitope peptide (e.g. one or more neoepitope peptides, two or more neoepitope peptides or three or more neoepitope peptides), and some of the neoepitope peptides have different amino acid sequences, or all of the neoepitope peptides have different amino acid sequences, the amino acid sequences may be different because:
the protein or peptide known or suspected to be expressed by a cancer cell in the subject are different; or
the protein or peptide known or suspected to be expressed by a cancer cell in the subject is the same, but the part of the protein or peptide known or suspected to be expressed by a cancer cell in the subject that the amino acid sequence of the neoepitope peptide corresponds to are different.

If the protein or peptide known or suspected to be expressed by a cancer cell in the subject is the same, but the part of the protein or peptide known or suspected to be expressed by a cancer cell in the subject that the amino acid sequence of the neoepitope peptide corresponds to are different, the parts may be different for one or more of the following reasons:
a longer part having the same at least one somatic mutated amino acid,
a shorter part having the same at least one somatic mutated amino acid, and/or
a part having the same at least one somatic mutated amino acid but the position of the mutated amino acid(s) is different in relation to the C- and N-terminus;
a different part of the same protein or peptide that has at least one different somatic mutated amino acid (for example, the protein or peptide has a frame shift mutation and the different parts are different parts of the frameshift mutated sequence of the protein or peptide).

In preferred embodiments, the amino acid sequences are different because the protein or peptide known or suspected to be expressed by a cancer cell in the subject is the same, but the part of the protein or peptide known or suspected to be expressed by a cancer cell in the subject that the amino acid sequence of the epitope peptide corresponds to are different because they are each different parts of a frameshift mutated sequence of the protein or peptide.

In preferred embodiments, the amino acid sequences are different because for each neoepitope peptide the protein or peptide known or suspected to be expressed by a cancer cell in the subject is different.

The Phagocytosable Particle and the Core of the Phagocytosable Particle

A phagocytosable particle for use according to the present invention is a particle able to be phagocytosed by a DC. It should be understood, however, that phagocytosable particles suitable for use in the present invention may be internalised by DCs via different routes (e.g. pinocytosis, clathrin-mediated endocytosis and non-clathrin-mediated endocytosis). Preferably, the phagocytosable particles are phagocytosable by a DC.

Antigens that are internalised into a DC by the phagocytic route are degraded in a non-uniform manner, which subsequently leads to a wider variety of antigen-derived peptides being presented by the DC.

Without wishing to be bound by theory, it is believed that DC cancer vaccines of the present invention are particularly effective at activating and expanding anticancer T-cells in vivo and ex-vivo because the antigenic constructs, when tightly associated to a phagocytosable particle as described herein, are phagocytosed by a DC, which subsequently leads to a wider variety of antigenic construct-derived peptides being presented by the DC, and thus greater activation and expansion of anticancer T-cells by the DCs contained in the DC cancer vaccine.

For a particle to be phagocytosed by a DC, the particle needs to be within a size range suitable to allow for phagocytosis. For example, a particle that is too small may not trigger phagocytosis by a DC, or a particle that is too large may not be phagocytosable by a DC. Complete phagocytosis leads to good antigen degradation by the DC and subsequently good presentation to T-cells via the MHC class I and II pathways. The optimal size has been investigated by the current inventors (see Examples 1 to 4).

A phagocytosable particle described herein comprises a core, and an antigenic construct tightly associated to the core. Preferably, at least one epitope peptide is a neoepitope peptide.

The size of the core needs to be within a range such that when the core is tightly associated to an antigenic construct(s), the core and the tightly associated antigenic construct(s) are phagocytosable by a DC. It is preferred that the size of the core is within a range such that when the core is tightly associated to a antigenic construct(s), the core and the tightly associated antigenic construct(s) are small enough that more than one phagocytosable particle can enter the same DC by phagocytosis. Having more than one phagocytosed particle in a DC maximises presentation of epitope(s) on the cell surface via the MHC class II pathway. Furthermore, it allows particles having different antigenic constructs to enter a DC, which means that the DC can present different epitopes from several particles in different phagosomes at the same.

In one preferred embodiment, the core has a largest dimension of less than 6 μm, less than 5.6 μm, less than 4 μm, less than 3 μm, less than 2.5 μm, less than 2 μm or less than 1.5 μm. More preferably the core has a largest dimension of less 1.5 μm. In another preferred embodiment, the core may have a largest dimension of greater than 0.001 μm, greater than 0.005 μm, greater than 0.01 μm, greater than 0.05 μm, greater than 0.1 μm, greater than 0.2 μm or greater than 0.5 μm. More preferably the core has a largest dimension of greater than 0.5 μm.

In one especially preferred embodiment, the core has a largest dimension in the range of 0.1 μm to 6 μm, for example 0.1 μm to 5.6 μm, 0.2 μm to 5.6 μm, 0.5 μm to 5.6 μm, 0.1 μm to 4 μm or 0.5 μm to 4 μm. More preferably, the core has a largest dimension in the range of 0.1 μm to 3 μm, for example, 0.5 μm to 3 μm, 0.2 μm to 2.5 μm, 0.5 μm to 2.5 μm, 0.2 μm to 2 μm, 0.5 μm to 2 μm or 1 μm to 2 μm. Even more preferably, the core has a largest dimension of about 1 μm, about 1.5 μm or about 2 μm. In a very preferred embodiment of the invention, the core is about 1 μm.

The core of the phagocytosable particle takes the form of any three-dimensional shape, for example any regular or irregular three-dimensional shape. Preferably, the phagocytosable particle is substantially spherical, in which case the dimensions of the phagocytosable particle refers to diameter.

The core of a phagocytosable particle may comprise a polymer, glass, ceramic material (e.g. the core may be a polymer particle, a glass particle or a ceramic particle). The material of the core may be a biodegradable and/or biocompatible material (e.g. the particle may be a biodegradable and/or biocompatible particle).

Preferably, the core comprises a polymer (for example, the core is a polymer particle). If the core comprises a polymer, it may be selected from the group consisting of a synthetic aromatic polymer (such as polystyrene e.g. the core is a polystyrene particle), a synthetic non-aromatic polymer (such as polyethylene, polylactic acid, poly(lactic-co-glycolic acid) and polycaprolactone, e.g. the core is a polyethylene particle, polylactic acid particle, poly(lactic-co-glycolic acid) particle or polycaprolactone particle), a naturally occurring polymer (such as collagen, gelatine, proteins (e.g. virus-like particles), lipids or albumin, e.g. the core is a collagen particle, gelatine particle or albumin particle), a polymeric carbohydrate molecule (such as a polysaccharide, for example agarose, alginate, chitosan or zymosan e.g. the core is an agarose particle, alginate particle, chitosan particle or zymosan particle).

In preferred embodiments, the core comprises polystyrene or polyethylene, and more preferably comprises polystyrene (e.g. the core is a polystyrene particle). Such polymers are biocompatible.

The present inventors have found that polystyrene particles are a particularly useful core for a phagocytosable particle for use in the present invention because they are nontoxic and are commercially available in various sizes and in various functionalisable forms. Furthermore, the present inventors have found phagocytosable particles comprising a polystyrene core, such as a polystyrene particle, are able to withstand stringent sterilisation procedures to prepare the particles for use in the preparation of a DC cancer vaccine of the invention. Such sterilisation procedures may include repeated washes with acid or alkali solutions and/or exposure to high temperatures.

In preferred embodiments, the core is a polystyrene particle with a largest dimension of less than 6 μm, preferably from about 1 μm to about 3 μm, and more preferably about 1 μm. Phagocytosable particles comprising a polystyrene particle core with a dimension of about 1 μm to about 3 μm, and especially about 1 μm, are efficiently phagocytosed by DCs and are also able to withstand stringent sterilisation procedures to remove pathogens (e.g. bacteria, fungus and viruses) and antigenic contaminants, such as pyrogens (e.g. endotoxins), which may be associated to the core or antigenic construct.

In preferred embodiments, the core has magnetic properties. For example, the core may have paramagnetic or superparamagnetic properties. Preferably, the core has superparamagnetic properties.

An example of a superparamagnetic core suitable for use with the invention are Dynabeads™ (Invitrogen). Dynabeads™ are available in various functionalisable forms, for example Dynabeads M-270 Carboxylic acid, Dynabeads M-270 Amine, and Dynabeads MyOne Carboxylic acid. Dynabeads™ are monosized superparamagnetic particles, which are composed of highly cross-linked polystyrene with evenly distributed magnetic material. The magnetic material may be iron oxide. Other examples of magnetic cores, in particular superparamagnetic cores, include Encapsulated Carboxylated Estapor® SuperParamagnetic Microspheres (Merck Chimie S.A.S.) and Sera-Mag SpeedBeads (hydrophilic) Carboxylate-Modified Magnetic particles (Cytiva). Encapsulated Carboxylated Estapor® SuperParamagnetic Microspheres are made of a core-shell structure which encapsulates an iron oxide core.

A phagocytosable particle for use in the present invention comprises an antigenic construct tightly associated to the core. An antigenic construct may be tightly associated to a core using a variety of means. For example, the antigenic construct may be attached to a core by a covalent bond, for example an amide bond between an amine group or a carboxylic acid group of the antigenic construct and a carboxylic acid group or an amine group on the surface of the core. Alternatively, an antigenic construct may be linked to a core via a metal chelate. For example, cores linked with a metal chelating ligand, such as iminodiacetic acid can bind metal ions such as Cu²⁺, Zn²⁺, Ca²⁺, Co²⁺ or Fe³⁺. These metal chelates can in turn bind proteins and peptides containing for example histidine or cysteine with great strength. Thus, cores with metal chelates can non-covalently bind to an antigenic construct. Preferably, the antigenic construct is covalently attached to the core. One example of associating the antigenic construct to the core is shown in Example 1.

A phagocytosable particle for use in the present invention comprises a core, and an antigenic construct tightly associated to the core. The phagocytosable particle may comprise one or more antigenic constructs associated to the core. For example, the phagocytosable particle may comprise one to 3 million antigenic constructs, preferably one to 2 million antigenic constructs, and more preferably one to 1 million antigenic constructs, for example one to 800,000 antigenic constructs, one to 500,000 antigenic constructs, one to 100,000 antigenic constructs, one to 10,000 antigenic constructs, one to 1000 antigenic constructs, one to 100 antigenic constructs, or one to 10 antigenic constructs; or for example 10 to 1 million, 100 to 1 million, 1000 to 1 million antigenic constructs, 10,000 to 1 million antigenic constructs, 100,000 to 1 million antigenic constructs, or 500,000 to 1 million antigenic constructs. Preferably a phagocytosable particle of the invention may comprise 500,000 to 1 million antigenic constructs.

In preferred embodiments, to maximise the delivery of antigenic construct into a DC (which can then be cleaved from the phagocytosable particle and processed by a DC, thus resulting in the presentation of a wide variety of epitope-derived peptides on the surface of the DC), a phagocytosable particle may comprise more than one (i.e. two or more, for example two to 3 million) antigenic constructs associated to the core. For example, the phagocytosable particle may comprise two to 1 million antigenic constructs tightly associated to a core (for example two to 800,000 antigenic constructs, two to 500,000 antigenic constructs, two to 100,000 antigenic constructs, two to 10,000 antigenic constructs, two to 1000 antigenic constructs, two to 100 antigenic constructs, or two to 10 antigenic constructs tightly associated to a core). Preferably, the phagocytosable particle comprises 10 or more antigenic constructs tightly associated to a core, such as 10 to 1 million antigenic constructs tightly associated to a core (for example 10 to 800,000 antigenic constructs, 10 to 500,000 antigenic constructs, 10 to 100,000 antigenic constructs, 10 to 10,000 antigenic constructs, 10 to 1000 antigenic constructs, or 10 to 100 antigenic constructs tightly associated to a core). More preferably, the phagocytosable particle comprises 100 or more antigenic constructs tightly associated to a core, such as 100 to 1 million antigenic constructs tightly associated to a core (for example 100 to 800,000 antigenic constructs, 100 to 500,000 antigenic constructs, 100 to 100,000 antigenic constructs, 100 to 10,000 antigenic constructs, or 100 to 1000 antigenic constructs tightly associated to a core). In certain embodiments, the phagocytosable particle comprises 1000 or more antigenic constructs tightly associated to a core, such as 1000 to 1 million antigenic constructs tightly associated to a core (for example 1000 to 800,000 antigenic constructs, 1000 to 500,000 antigenic constructs, 1000 to 100,000 antigenic constructs, or 1000 to 10,000 antigenic constructs tightly associated to a core). In certain embodiments, the phagocytosable particle comprises 10,000 or more antigenic constructs tightly associated to a core, such as 10,000 to 1 million antigenic constructs tightly associated to a core (for example 10,000 to 800,000 antigenic constructs, 10,000 to 500,000 antigenic constructs, or 10,000 to 100,000 antigenic constructs tightly associated to a core). In certain embodiments, the phagocytosable particle comprises 100,000 or more antigenic constructs tightly associated to a core, such as 100,000 to 1 million antigenic constructs tightly associated to a core (for example 100,000 to 800,000 antigenic constructs or 100,000 to 500,000 neoantigenic constructs tightly associated to a core). In one preferred embodiment, the phagocytosable particle comprises 500,000 or more antigenic constructs tightly associated to a core, such as 500,000 to 1 million antigenic constructs tightly associated to a core, or 500,000 to 2 million antigenic constructs tightly associated to a core, or 500,000 to 3 million antigenic constructs tightly associated to a core. In other embodiments, the phagocytosable particle comprises more than 1 million antigenic constructs tightly associated to a core, for example, 1 million to 3 million antigenic constructs, or 1 million to 2 million antigenic constructs tightly associated to a core.

In embodiments where the phagocytosable particle of the invention comprises more than one antigenic construct associated to the core (for example, 2 or more, 10 or more, 100 or more, 1000 or more, 10,000 or more, 100,000 or more, or 500,000 or more neoantigenic constructs associated to the core), the antigenic constructs associated to the core may be the same, or may be different (i.e. some or all of the antigenic constructs associated to the core may be different). They may be different by comprising different epitope peptide sequences or they may be different by comprising a different combination of epitope peptides. They may alternatively, or additionally, be different by comprising one or more different spacer moieties or further amino acid sequences, if such moieties and sequences are present. Antigenic constructs that are different may be referred to as “different types” of antigenic construct. Antigenic constructs that are the same may be referred to as the “same type” of antigenic construct.

A phagocytosable particle comprising more than one antigenic construct associated to the core (for example, two or more, 10 or more, 100 or more, 1000 or more, 10,000 or more, 100,000 or more, or 500,000 or more antigenic construct associated to the core) can comprise one type of antigenic construct tightly associated to a core (i.e. all the antigenic construct tightly associated to the core are the same). In one embodiment of the invention, a phagocytosable particle comprises 100,000 to 1 million antigenic constructs tightly associated to a core, wherein the 100,000 to 1 million antigenic constructs are the same type of antigenic construct.

In alternative embodiments, a phagocytosable particle comprising more than one antigenic construct associated to the core (for example, two or more, 10 or more, 100 or more, 1000 or more, 10,000 or more, 100,000 or more, or 500,000 or more antigenic constructs associated to the core) can comprise two different antigenic construct types tightly associated to a core. In other embodiments, a phagocytosable particle comprising more than 10 antigenic constructs associated to the core (for example, 100 or more, 1000 or more, 10,000 or more, 100,000 or more, or 500,000 or more antigenic constructs associated to the core) can comprise two or more different antigenic construct types tightly associated to a core. For example such a phagocytosable particle may comprise two to 10 different antigenic construct types tightly associated to a core (for example, 2, 3, 4, 5, 6, 7, 8, 9 or 10 different antigenic construct types). Preferably, such a phagocytosable particle may comprises two to six different antigenic constructs types (for example, 2, 3, 4, 5 or 6 different antigenic construct types). In one embodiment of the invention, a phagocytosable particle comprises 100,000 to 1 million antigenic constructs tightly associated to a core, wherein the 100,000 to 1 million antigenic constructs comprise two or more different antigenic construct types. For example, two to six different antigenic construct types (for example 2, 3, 4, 5 or 6 different antigenic construct types).

In one embodiment, the phagocytosable particle further comprises an adjuvant tightly associated to the core. The term “adjuvant” as used herein is to be understood as any substance that enhances an immune response towards an antigen. Particular examples of adjuvants include dsRNA analogues, such as polyinosinic:polycytidylic acid, Incomplete Freund's Adjuvant, cytokines (for example, IL2, IL4, IL17 and IL15), CD40, keyhole limpet hemocyanin, Toll-like receptors, CpG oligodeoxynucleotides, saponins, colloidal alum, and analogues of lipid A of lipopolysaccharide. Adjuvants may be tightly associated to the core in the same manner as that described herein for tightly associating an antigenic construct to a core.

Sterilisation of the Phagocytosable Particles

The present inventors have advantageously found that phagocytosable particles comprising a core and an antigenic construct tightly associated to the core, may be efficiently washed and sterilised before contacting a DC according to the method of the present invention. This is particularly advantageous because the washed and sterilised phagocytosable particles comprise lower levels of pathogens (e.g. bacteria, fungus and viruses) and contaminants, such as endotoxins (e.g. lipopolysaccharides) and other antigenic contaminants. Such contaminants can elicit non-specific immune responses in the subject. Washing and sterilising phagocytosable particles before contacting a DC therefore provides a safer DC cancer vaccine.

In preferred embodiments, the phagocytosable particle for use in the invention comprises a magnetic core, for example a paramagnetic or superparamagnetic core. A phagocytosable particle comprising a magnetic core, can be collected and/or held in place by a magnet. It is also possible to perform a wash by other means, such as by holding the phagocytosable particles (whether paramagnetic or not) in a column, or sedimenting the particles by gravity or by centrifugation.

The particular manner of the wash is not critical in the context of the present invention. For instance, the wash may involve subjecting a phagocytosable particle to a high pH, to a low pH, to a high temperature, to a sterilising/denaturing agent or a combination thereof

The wash may involve subjecting the phagocytosable particle to alkali, preferably a strong alkali, for example at least 0.1M, 0.5M, 1M, 2M, 3M, 4M, 5M, 6M, 7M or 8M alkali. Preferably, the wash may involve subjecting the phagocytosable particle to at least 1M sodium hydroxide (NaOH), for example at least 2M NaOH. Preferably, the wash involves subjecting the phagocytosable particle to a high pH of at least 13.0, more preferably at least 14.0, most preferably at least 14.3. Other alkalis that may be used include, but are not limited to: lithium hydroxide (LiOH), potassium hydroxide, (KOH), rubidium hydroxide (RbOH), cesium hydroxide (CsOH), magnesium hydroxide (Mg(OH)₂), calcium hydroxide (Ca(OH)₂), strontium hydroxide (Sr(OH)₂), and barium hydroxide (Ba(OH)₂). Preferably, the wash involves subjecting the phagocytosable particle to a high pH of at least 13.0, more preferably at least 14.0, most preferably at least 14.3.

The wash may also involve subjecting the phagocytosable particle to an acid, preferably a strong acid, for example at least 0.1M, 0.5M, 1M, 2M, 3M, 4M, 5M, 6M, 7M or 8M acid. Preferably, the wash may involve subjecting the phagocytosable particle to at least 1M hydrochloric acid (HCl), for example at least 2M HCl. Other acids that may be used include, but are not limited to: hydroiodic acid (HI), hydrobromic acid (HBr), perchloric acid (HClO₄), nitric acid (HNO₃) and sulfuric acid (H₂SO₄). The wash may also involve subjecting the phagocytosable particle to further sterilising/denaturing agents, such as urea and/or guanidine-HCl.

Preferably, the wash results in the phagocytosable particle being aseptic and/or sterile. More preferably, the wash results in the phagocytosable particle being sterile. “Aspetic” is defined herein as being free from disease-causing microorganisms and viruses. “Sterile” is defined herein as being free from all biological contaminants.

Preferably, the wash also removes antigenic contaminants such as pyrogens (e.g. endotoxins) from the phagocytosable particle. Preferably, the wash provides the phagocytosable particle with an endotoxin contamination of less than 100 pg/ml, preferably less than 50 pg/ml, more preferably less than 25 pg/ml and most preferably less than 10 pg/ml.

Thus, in preferred embodiments, the phagocytosable particle is sterile and has an endotoxin contamination of less than 100 pg/ml.

The wash may comprise a single wash or several repeated washes, such as 2, 3, 4 or 5 washes. In addition, or alternatively, the phagocytosable particle may be subjected to a high temperature, such as at least 90° C., preferably at least 92° C., more preferably at least 95° C., for example at least 100° C. or at least 110° C.

The present inventors have advantageously found that when an antigenic construct is associated to a core by a covalent bond, the phagocytosable particle can withstand stringent sterilisation and washing procedures that reduce the amount of antigenic contaminants, such as pyrogens (e.g. endotoxins), that may be bound to an antigenic construct or core. This means that the phagocytosable particles described herein are especially suitable for use in a method of preparing a DC cancer vaccine according to the present invention.

A Plurality of Phagocytosable Particles

Phagocytosable particles having different cores (e.g. cores having different sizes and/or comprising different materials/polymers as described herein) are referred to herein as phagocytosable particles of a “different set”. Phagocytosable particles having the same core (e.g. cores having the same size and comprising the same materials/polymer) may be referred to herein as phagocytosable particles of the “same set”.

Phagocytosable particles that have the same core (i.e. they are of the same phagocytosable particle set), but that are different due to having different types of antigenic construct tightly associated to the core, are referred to herein as phagocytosable particles of “different groups”. Phagocytosable particles that have the same core and the same type of antigenic constructs tightly associated to the core are referred to herein as phagocytosable particles of the “same group”.

In one embodiment, a plurality of phagocytosable particles comprises one phagocytosable particle set which consists of one phagocytosable particle group (i.e. all of the phagocytosable particles in the composition are the same).

Alternatively, a plurality of phagocytosable particles can comprise one phagocytosable particle set which comprises two or more different groups of phagocytosable particle (for example, 2, 3, 4, 5, 6, 7, 8, 9, 10, 12, 15, 20, 25, 30, 40 or 50, or more, phagocytosable particle groups).

In another embodiment, a plurality of phagocytosable particles can comprise one phagocytosable particle set which comprises 2 to 50 different groups of phagocytosable particle (for example, 2, 3, 4, 5, 6, 7, 8, 9, 10, 12, 15, 18, 20, 25, 30, 40 or 50 phagocytosable particle groups).

In another embodiment, a plurality of phagocytosable particles can comprise one phagocytosable particle set which comprises 2 to 30 different groups of phagocytosable particle (for example, 2, 3, 4, 5, 6, 7, 8, 9, 10, 12, 15, 18, 20, 25, or 30 phagocytosable particle groups).

In another embodiment, a plurality of phagocytosable particles can comprise one phagocytosable particle set which comprises 2 to 20 different groups of phagocytosable particle (for example, 2, 3, 4, 5, 6, 7, 8, 9, 10, 12, 15, 18 or 20 phagocytosable particle groups).

In another embodiment, a plurality of phagocytosable particles can comprise one phagocytosable particle set which comprises 2 to 15 different groups of phagocytosable particle (for example, 2, 3, 4, 5, 6, 7, 8, 9, 10, 12, or 15 phagocytosable particle groups).

In another embodiment, a plurality of phagocytosable particles can comprise one phagocytosable particle set which comprises 2 to 10 different groups of phagocytosable particle (for example, 2, 3, 4, 5, 6, 7, 8, 9, or 10 phagocytosable particle groups).

In another embodiment, a plurality of phagocytosable particles can comprise one phagocytosable particle set which comprises 2 to 8 different groups of phagocytosable particle (for example, 2, 3, 4, 5, 6, 7, or 8 phagocytosable particle groups).

In another embodiment, a plurality of phagocytosable particles can comprise one phagocytosable particle set which comprises 2 to 6 different groups of phagocytosable particle (for example, 2, 3, 4, 5, or 6 phagocytosable particle groups).

In certain embodiments, a plurality of phagocytosable particles can comprise two or more different phagocytosable particle sets (i.e. each set having different cores, for example, each set having cores of different sizes and/or comprising different materials as described herein) and each set can consist of one phagocytosable particle group. For example, the a plurality of phagocytosable particles can comprise 2, 3, 4 or 5 phagocytosable particle sets and each set can consist of one phagocytosable particle group. Preferably, the plurality of phagocytosable particles can comprise 2 or 3 phagocytosable particle sets and each set consists of one phagocytosable particle group.

In other embodiments, a plurality of phagocytosable particles can comprise two or more different phagocytosable particle sets (for example, 2, 3 or 4 phagocytosable particle sets) and each set can comprise of two or more different phagocytosable particle group (for example 2, 3, 4, 5, 6, 7, 8, 9, 10, 12, 15, 20, 25 or 30 phagocytosable particle groups).

In other embodiments, a plurality of phagocytosable particles can comprise two or more different phagocytosable particle sets (for example, 2, 3 or 4 phagocytosable particle sets) and each set can comprise 2 to 30 different groups of phagocytosable particle (for example 2, 3, 4, 5, 6, 7, 8, 9, 10, 12, 15, 18, 20, 25 or 30 phagocytosable particle group).

In other embodiments, a plurality of phagocytosable particles can comprise two or more different phagocytosable particle sets (for example, 2, 3 or 4 phagocytosable particle sets) and each set can comprise 2 to 20 different groups of phagocytosable particle (for example 2, 3, 4, 5, 6, 7, 8, 9, 10, 12, 15, 18 or 20 phagocytosable particle groups).

In other embodiments, a plurality of phagocytosable particles can comprise two or more different phagocytosable particle sets (for example, 2, 3 or 4 phagocytosable particle sets) and each set can comprise 2 to 15 different groups of phagocytosable particle (for example 2, 3, 4, 5, 6, 7, 8, 9, 10, 12, or 15 phagocytosable particle groups).

In other embodiments, a plurality of phagocytosable particles can comprise two or more different phagocytosable particle sets (for example, 2, 3 or 4 phagocytosable particle sets) and each set can comprise 2 to 10 different groups of phagocytosable particle (for example 2, 3, 4, 5, 6, 7, 8, 9, or 10 phagocytosable particle groups).

In other embodiments, a plurality of phagocytosable particles can comprise two or more different phagocytosable particle sets (for example, 2, 3 or 4 phagocytosable particle sets) and each set can comprise 2 to 8 different groups of phagocytosable particle (for example 2, 3, 4, 5, 6, 7, or 8 phagocytosable particle groups).

In other embodiments, a plurality of phagocytosable particles can comprise two or more different phagocytosable particle sets (for example, 2, 3 or 4 phagocytosable particle sets) and each set can comprise 2 to 6 different groups of phagocytosable particle (for example 2, 3, 4, 5, or 6 phagocytosable particle groups).

For the avoidance of doubt, in embodiments having more than one group of phagocytosable particle and more than one set of phagocytosable particle, each group of a phagocytosable particle set is independent from each group of another phagocytosable particle set. Thus, a group from one phagocytosable particle set can have the same antigenic construct type as a group from another phagocytosable particle set. Alternatively, a group from one phagocytosable particle set can have antigenic constructs that are a different type to those of a group from another phagocytosable particle set.

Dendritic Cells

The DC sample used in the present invention (i.e. the DC sample provided in step (ii) of the present invention) is preferably harvested from a subject with a cancer. A DC sample may also be harvested from a different subject, such as a healthy subject. If the DC sample is obtained from a different subject, it is preferably obtained from the same species and donor-matched with respect to MHC receptors. However, use of genetically engineered DCs from a different species is also envisioned. More preferably, the DC sample is obtained from the same subject. If the DC sample is derived from the same subject, any potential for a mismatch between the DCs and the subject is avoided.

In certain embodiments, a DC sample may be derived from peripheral blood from a subject. Typically, the DC sample is obtained from a subject by leukapheresis to provide a leukapheresis product, allowing other cell types such as red blood cells and thrombocytes to be return to the patient. Leukapheresis is a routine method employed for harvesting various types of blood derived cell products. Monocytes may be purified from a leukapheresis derived peripheral blood mononuclear cell (PBMC) product by various methods, such as immune affinity purification or by counter flow centrifugation. Using leukapheresis to obtain a DC sample for use in the invention is particularly desirable because it is particularly compatible with the production of a DC cancer vaccine under Good Manufacturing Practices (GMP). Alternatively, monocytes may also be purified from PBMC isolated by density gradient centrifugation of whole peripheral blood, or fractions thereof, from a subject.

A PBMC sample generally consists of lymphocytes (70-90%) and monocytes (10-30%), while red blood cells, granulocytes and plasma have been removed. Monocytes may in some instances make up 10% to 20% of the cell numbers in a PBMC sample, for example 10% to 15%. Obtaining PBMCs from peripheral blood samples can be achieved using routine protocols. A PBMC sample may be freshly used or subjected to freezing. The possibility of using frozen cells is of great practical advantage from a logistical point of view.

DCs derived from monocytes may be “immature” or “mature” DCs.

Mature and immature DCs may be readily distinguished by phenotypic differences. For example, immature DCs are generally characterised by high phagocytic activity and low level expression of costimulatory molecules and immunostimulatory cytokines. In contrast, mature DCs generally express high levels of costimulatory molecules and immunostimulatory cytokines, which make them potent activators of T-cells. Mature DCs may also be distinguished from immature DCs by the presence of certain cell surface markers, in particular by the presence of CD83 and CD86.

The DC cancer vaccine of the present invention may comprise immature and/or mature DCs. Preferably, the DC cancer vaccine comprises predominantly mature DCs.

Methods for generating DCs from monocytes generally include culturing the cells with an appropriate cytokine(s). An established method of generating immature DCs from monocytes involves culturing monocytes with a mixture of IL4 and granulocyte-macrophage colony-stimulating factor (GM-CSF). Mature DC may be generated from immature DCs. Methods of maturing DCs for use in a DC cancer vaccine of the invention include incubating immature DCs with a maturation cocktail, such as a mixture of TNFα, IL1β, IL6, and optionally, prostaglandin E2 (PGE2). Additional cocktails include various mixtures of cytokines (for example, TNFα, IL1β, IFNα, IFNγ) and TLR agonists (for example polyinosinic:polycytidylic acid (poly I:C), lipopolysaccharide, lipid A of lipopolysaccharide and analogues thereof, resiquimod). Specific examples of maturations cocktails include, a mixture of TNFα, IL1β, IFNα, IFNγ and poly I:C; a mixture of IFNγ and poly I:C; a mixture of IFNγ and resiquimod; or a mixture of IFNγ, resiquimod, poly I:C and lipopolysaccharide (see for example Lovgren et al., Cancer Immunol Immunother (2017) 66:1333-1344, which is incorporated herein by reference).

A skilled person in the art can readily determine the effective amounts of the individual reagents (e.g. cytokines) required to generate immature DCs from monocyte cells, and the effective amounts of the individual components of a maturation cocktail (e.g. cytokines and/or TLR agonists) required to provide mature DCs. The particular method used for obtaining or generating immature DCs, and the method of generating mature DCs from immature DCs, is not critical in the context of the present invention.

Kits of the Invention

The present invention further provides a kit comprising phagocytosable particle as described herein and reagents suitable for use in generating DCs from a blood sample from a subject. For example, suitable reagent may include those commonly found in DC maturation cocktails (for example, one or more of TNFα, IL1β, IFNα, IFNγ and TLR agonists, such a poly I:C, lipid A of lipopolysaccharide and analogues thereof, and resiquimod). The kit may further comprise reagents for assisting in expanding a T-cell population in vivo and/or in vitro, such as IL2. The kit may optionally also comprise IL7 and/or IL15. The kit may further comprise reagents for sterilising the phagocytosable particles as described herein.

The invention further provides a kit comprising a core as described herein, together with coupling reagents for coupling one or more antigenic constructs describe herein to the core. The kit may further comprise one or more antigenic constructs or reagents for producing antigenic constructs, for example ready-to-use vectors adjusted for different cloning and expression conditions, or that comprise a cDNA sequence encoding one or more antigenic constructs.

Details of the Method Steps

The present invention provides a method for the manufacture of a DC cancer vaccine. The method of the invention is carried out in vitro. For the avoidance of doubt, the plurality of phagocytosable particles provided in step (i) of the invention may independently have any of the properties and/or characteristics of the phagocytosable particles described herein.

The DC sample provided in step (ii) of the invention contains one or more DC. The DC sample may comprise one or more immature DC and/or one or more mature DC. Preferably, the DC sample provided in step (ii) comprises predominantly immature DCs.

The DC sample provided in step (ii) is contacted with a plurality of phagocytosable particles in vitro and under conditions allowing for the phagocytosis of at least one phagocytosable particle by a DC.

In embodiments where the sample provided in step (ii) is a sample of immature DCs, the method may comprise an additional step before step (iii) which comprises the maturation of the immature DCs to form mature DCs. Additionally, or alternatively, the method may comprise an additional step after step (iii) which comprises the maturation of the immature DCs to mature DCs. Additionally, or alternatively, step (iii) of the present invention may comprise contacting the immature DCs with a plurality of phagocytosable particles and simultaneously maturing the immature DCs to form mature DCs.

In certain embodiments, the DC sample provided in step (ii) is a sample of mature DCs, and in step (iii), the sample of mature DC is contacted with a plurality of phagocytosable particles in vitro and under conditions allowing for the phagocytosis of at least one phagocytosable particle by a mature DC.

Typically, the DC sample provided in step (ii) comprises one to about 10⁹ DCs. For example, one to about 10⁵ DCs; or, for example, about 10⁵ to about 10⁶ DCs or about 10⁵ to about 10⁹ DCs; or, for example, about 10⁶ to about 10⁷ DCs; or for example, about 10⁷ to about 10⁸ DCs, or for example, about 10⁸ to about 10⁹ DCs.

In embodiments, the DC sample is contacted with 100 to 1×10⁹ phagocytosable particles, for example 100 to 1×10⁸ phagocytosable particles, 100 to 1×10⁷ phagocytosable particles or 1000 to 1×10⁷ phagocytosable particles. Typically, the ratio of phagocytosable particles to DCs is in the range about 1000:1 to about 1:10. For example, about 1000:1 to about 1:1, about 1000:1 to about 3:1, about 500:1 to about 1:1, about 250:1 to about 1:1, about 100:1 to about 1:1, about 50:1 to about 1:1, about 50:1 to about 3:1, about 40:1 to about 3:1, about 30:1 to about 3:1, about 20:1 to about 3:1, or about 20:1 to about 10:1 (for example, about 500:1, about 250:1, about 100:1, about 50:1, about 40:1, about 20:1 or about 10:1). The ratio can be optimised depending on the size of the phagocytosable particle. For example, for a phagocytosable particle with a largest diameter of about 1 μm, the ratio may be in the range 50:1 to 2:1, for example 25:1 to 5:1, 15:1 to 7:1, and 10:1 of phagocytosable particles to DCs. The present inventors have found that a ratio of phagocytosable particles to DCs of about 40:1 to about 3:1 is especially effective at providing a DC cancer vaccine that is effective at eliciting a strong anticancer T-cell response. Thus, in certain embodiments, the DC sample is contacted phagocytosable particles at a ratio of phagocytosable particle to DCs of about 40:1 to about 3:1.

In certain embodiments, step (iii) further comprises contacting the sample of DCs with a PDL1 inhibitor. Without wishing to be bound by theory, the present inventors believe that the addition of a PDL1 inhibitor to step (iii) of the method is effective at blocking the immunosuppressive effective of PDL1 expressed by the DCs, thus resulting in a stronger and more robust activation of anticancer T-cells by the DC cancer vaccine of the invention.

In certain embodiments, the method for the manufacture of a DC cancer vaccine of the invention further comprises the steps of:
(v) removing extracellular phagocytosable particles from the sample of DCs; and/or
(vi) isolating at least one DC containing at least one phagocytosable particle from the sample of DCs in step (iii) of the present invention.

Preferably, step (v) involves the positive selection of a DC containing at least one phagocytosable particle by means of a magnet or magnetic field. That is to say that a DC containing at least one phagocytosable particle is isolated from the other components of the sample by means of a magnet or magnetic field. Isolation of the DC by means of a magnet or magnetic field provides a sample comprising predominately DCs that have phagocytosed at least one phagocytosable particle described herein.

For the avoidance of doubt, as will be evident from the foregoing, it is a particular aspect of the present invention that the present invention provides a use of a phagocytosable particle described herein in the preparation of a DC cancer vaccine of the present invention.

Formulations

The present invention provides a DC cancer vaccine produced by the method of the present invention. A DC cancer vaccine of the present invention comprises one or more DCs that contain one or more phagocytosable particles described herein. Preferably, the DC cancer vaccine of the present invention is in a form suitable for injecting into a subject.

Suitable formulations for use in the present invention generally comprise a suspension of DCs in an appropriate physiologically acceptable medium. Such mediums are known in the art and include, for example, aqueous sterile injectable solutions (e.g. saline, such as PBS), cell culture mediums suitable for clinical use and autologous plasma, which may also contain anti-oxidants, buffering agent (e.g. sodium phosphate, potassium phosphate, TRIS and TEA), bacteriostats, and solutes which render the DC cancer vaccine dose isotonic with the blood of the intended recipient.

Preferably, the formulation is an injectable pharmaceutical composition. More preferably, the formulation is suitable for subcutaneous, intradermal, intramuscular, intravenous (bolus or infusion), intratumorally, intraarticular and intralymphatic administration, although the most suitable route may depend upon, for example, the type of cancer or tumour present in the subject. Even more preferably, the injectable composition is suitable for subcutaneous or intradermal administration.

It should be understood that in addition to the ingredients particularly mentioned above, the pharmaceutically acceptable formulations of the invention may include other agents conventional in the art having regard to the type of formulation in question.

Whilst the DCs for use in the various embodiments of the present invention may be used as the sole active ingredient, it is also possible for the DCs to be used in combination with one or more further active agents. Thus, the invention also provides a DC cancer vaccine for use in the treatment or prophylaxis of cancer in a subject, or for use in methods of treatment or prophylaxis of cancer in a subject, together with a further active agent, for simultaneous, sequential or separate administration. Such further active agents may be agents useful in the treatment of cancer, or other pharmaceutically active materials, but are preferably agents known for the treatment of cancer. Such agents are known in the art. Examples of further active agents include alkylators, antimetabolites, anti-tumour antibiotics, histone deacetylase inhibitors, immunomodulatory drugs, microtubule interactive drugs, protein kinase inhibitors, steroids, topoisomerase inhibitors, cell cycle inhibitors, and angiogenesis inhibitors.

Thus, the DC cancer vaccine of the present invention may be administered together with one or more compounds known for the treatment or prophylaxis of cancer, for example one or more alkylator, antimetabolite, anti-tumour antibiotic, histone deacetylase inhibitor, immunomodulatory drug, microtubule interactive drugs, protein kinase inhibitor, steroid, topoisomerase inhibitor cell cycle inhibitor, or angiogenesis inhibitor. Preferably, the one or more compounds is an immunomodulatory drug. More preferably, the immunomodulatory drug is a checkpoint inhibitor. Suitable checkpoint inhibitors include antibodies, antibody fragments, peptides, or small molecules. For example, antibodies, antibody fragments, peptides, or small molecules that inhibit or modulate PD1, PDL1 or CTLA4 activity.

In preferred embodiments, the DC cancer vaccine of the present invention comprises a PDL1 inhibitor, which is motivated by the fact that DCs derived from monocytes often express the PDL1 ligand, known to bind to the checkpoint molecule PD-1 expressed on the anti-tumour T-cells, thereby inhibiting the efficient activation of the T-cells to eliminate the tumour cells.

When used in a combination, the precise dosage of the further active agent(s) and/or adjuvant(s) will vary with the dosing schedule, the oral potency of the particular agent chosen, the age, size, sex and condition of the subject (typically mammal or human), the nature and severity of the cancer, and other relevant medical and physical factors. Thus, a precise therapeutic dose or booster dose cannot be specified in advance and can be readily determined by the caregiver or clinician. An appropriate amount can be determined by routine experimentation from animal models and human clinical studies. For humans, a therapeutic or booster dose will be known or otherwise be determined by one of ordinary skill in the art.

The individual components of such combinations can be administered separately at different times during the course of therapy or concurrently in divided or single combination forms. The present invention is therefore to be understood as embracing all such regimes of simultaneous or alternating treatment.

The above further active agent(s), when employed in combination with compounds useful in the invention, may be used, for example, in those amounts indicated in the Physicians' Desk Reference (PDR) or as otherwise determined by one of ordinary skill in the art.

Treatments

The present invention provides a DC cancer vaccine for use in the treatment or prophylaxis of cancer in a subject. The present invention also provides methods of treating or preventing cancer in a subject comprising the step of administering to the subject a DC cancer vaccine of the invention. The present invention also provides a use of the DCs described herein for the manufacture of a medicament for the treatment or prophylaxis of cancer.

The subject may have a cancer that may be classed as refractory, relapsed or refractory-relapsed. Additionally, or alternatively, the subject may have a cancer that is partially or completely resistant to chemotherapy. Additionally, or alternatively, the subject may have a cancer that has a primary resistance to treatment with immune checkpoint inhibitors, or a cancer that has acquired resistance to an immune checkpoint inhibitor after first having been treated with an immune checkpoint inhibitor.

The subject may have previously received one or more doses of a chemotherapeutic agent. For example, the subject may have received one or more doses of a chemotherapeutic agent as treatment for a cancer. Additionally or alternatively, the subject may have undergone lymphodepletion (i.e. induced reduction of lymphocyte levels) by administering one or more doses of a chemotherapeutic agent to the subject before administering the anticancer T-cells prepared in vitro according to the invention.

The DC cancer vaccine of the invention is capable of eliciting a robust anticancer immune response towards a cancer cell in a subject. On a general level, immune responses can be categorised as either an innate immune response or an adaptive immune response. An innate immune response is an immune response that is not intrinsically affected by prior contact with an antigen. Such a response is often characterised by the activation and expansion of naïve T-cells and B-cells. In contrast, an adaptive immune response is an immune response that requires prior contact with an antigen. The adaptive immune response generally follows shortly after an innate immune response, and ultimately leads to immunological memory towards an antigen. When the immune system comes into contact with an antigen for the first time, the immune response that is elicited (innate and adaptive responses) is often referred to as a “primary immune response”. Later activation of the memory T-cells and B-cells by the same antigen results in a rapid and specific immune response towards the antigen, this rapid and specific immune response towards the antigen is often referred to as a “secondary immune response”. A particular advantage of the DC cancer vaccine of the present invention is that it elicits a robust anticancer immune response by activating both the innate and adaptive immune response in a subject.

The treatments of the invention may be used to treat or prevent any form of cancer, for example a solid cancer, a metastatic solid cancer or a hematologic malignancy.

A “solid cancer” herein is, for example, an abnormal mass of tissue that originates in an organ. The solid cancer may be malignant. Different types of solid cancers are named for the type of cells that form them. Types of solid cancer include sarcomas, carcinomas, and lymphomas. Examples of solid cancers include adrenal cancer, anal cancer, anaplastic large cell lymphoma, angioimmunoblastic T-cell lymphoma, B-cell lymphoma, bile duct cancer, urinary bladder cancer, brain/CNS tumours, breast cancer, cervical cancer, colon cancer, head and neck cancers, endometrial cancer, oesophagus cancer, ewing family of tumours, eye cancer, gallbladder cancer, gastrointestinal carcinoid tumours, gastrointestinal stromal tumour (gist), gestational trophoblastic disease, hepatosplenic T-cell lymphoma, Hodgkin's lymphoma, intravascular large B-cell lymphoma, kidney cancer, laryngeal and hypopharyngeal cancer, liver cancer, lung cancer (non-small cell and small cell), lung carcinoid tumour lymphomatoid granulomatosis, malignant mesothelioma, nasal cavity and paranasal sinus cancer, nasopharyngeal cancer, neuroblastoma, nodal marginal zone B cell lymphoma, non-Hodgkin's lymphoma, oral cavity and oropharyngeal cancer, osteosarcoma, ovarian cancer, pancreatic cancer, penile cancer, pituitary tumours, primary effusion lymphoma, prostate cancer, retinoblastoma, rhabdomyosarcoma, salivary gland cancer, sarcoma, skin cancer (basal and squamous cell, melanoma and merkel cell), small intestine cancer, stomach cancer, testicular cancer, thymus cancer, thyroid cancer, uterine sarcoma, vaginal cancer, vulvar cancer, Waldenstrom macroglobulinemia, and Wilms' tumour. The treatments of the invention are especially effective in the treatment of solid cancers. As such, the subject of the invention may have a solid cancer. The treatments of the invention are particularly effective in the treatment of solid cancers selected from the group consisting of: anal cancer, urinary bladder cancer, breast cancer, cervical cancer, colon cancer, liver cancer, lung cancer (non-small cell and small cell), lung carcinoid tumour, ovarian cancer, pancreatic cancer, penile cancer, prostate cancer, stomach cancer, testicular cancer, uterine sarcoma, vaginal cancer, vulvar cancer, and even more especially for the treatment of breast cancer, colon cancer, liver cancer, lung cancer (non-small cell and small cell), lung carcinoid tumour, pancreatic cancer, prostate cancer, ovarian cancer and urinary bladder cancer.

The treatments of the invention are also especially effective in the treatment of metastatic solid cancers. Metastatic cancer is cancer which has spread from the primary site of origin into one or more different areas of the body.

The cancer may alternatively be any form of hematologic malignancy. A hematologic malignancy is a form of cancer that begin in the cells of blood-forming tissue, such as the bone marrow, or lymphatic system. In many hematologic malignancies, the normal blood cell development process is interrupted by uncontrolled growth of an abnormal type of blood cell. Examples of hematologic cancer include leukaemia, lymphomas, myelomas and myelodysplastic syndromes (lymphomas may be classed as both a solid cancer and a hematologic malignancy). Examples of hematologic malignancies include acute basophilic leukaemia, acute eosinophilic leukaemia, acute erythroid leukaemia, acute lymphoblastic leukaemia, acute megakaryoblastic leukaemia, acute monocytic leukaemia, acute myeloblastic leukaemia with maturation, acute myelogenous leukaemia, acute myeloid dendritic cell leukaemia, acute promyelocytic leukaemia, adult T-cell leukaemia/lymphoma, aggressive NK-cell leukaemia, anaplastic large cell lymphoma, and plasmacytoma, angioimmunoblastic T-cell lymphoma, B-cell chronic lymphocytic leukaemia, B-cell leukaemia, B-cell lymphoma, B-cell prolymphocytic leukaemia, chronic idiopathic myelofibrosis, chronic lymphocytic leukaemia, chronic myelogenous leukaemia, chronic myelomonocytic leukaemia, chronic neutrophilic leukaemia, extramedullary, hairy cell leukaemia, hepatosplenic T-cell lymphoma, Hodgkin's lymphoma, intravascular large B-cell lymphoma, Kahler's disease, lymphomatoid granulomatosis, mast cell leukaemia, multiple myeloma, myelomatosis, nodal marginal zone B cell lymphoma, non-Hodgkin's lymphoma, plasma cell leukaemia, primary effusion lymphoma, and Waldenstrom macroglobulinemia.

Dosing and Dosage Regimens

A suitable dose of the DC cancer vaccine of the invention is a dose sufficient to elicit an immune response against a cancer in a subject. For example, a primary immune response and/or a secondary immune response.

In certain embodiments, the use of a DC cancer vaccine of the invention for the treatment or prophylaxis of cancer comprises administering a therapeutic dose of the DC cancer vaccine to a subject. In certain embodiments, the method of treatment or prophylaxis of cancer comprises administering a therapeutic dose of a DC cancer vaccine to a subject.

The dose of a DC cancer vaccine of the invention required to treat or prevent a cancer in a subject, or induce the in vivo expansion of anticancer T-cells in a subject, will vary with the route of injection and the characteristics of the subject under treatment, for example the species, age, weight, sex, medical conditions, the particular cancer and its severity, and other relevant medical and physical factors. An ordinarily skilled physician can readily determine and administer the effective amount of DCs required for treatment or prophylaxis of a cancer.

A therapeutic dose may be administered as single unit dosage that comprises a therapeutic dose of the DC cancer vaccine of the invention, or as multiple unit dosages when a unit dosage comprise a fraction of a therapeutic dose.

In certain preferred embodiments, a subject is administered a therapeutic dose of the DC cancer vaccine and is then administered at least one further (or “subsequent”) therapeutic dose of the DC cancer vaccine. Further (or “subsequent”) therapeutic doses of the DC cancer vaccine may be administered daily, every second or third day, weekly, every second, third or fourth week, monthly, every second, third or fourth month, every 6 months, or every year. The number and frequency of further therapeutic dose(s) of the DC cancer vaccine will depend on the subject, and the form and severity of the cancer to be treated.

For example, the use of the DC cancer vaccine of the invention for the treatment or prophylaxis of cancer may comprise administering one or more subsequent therapeutic doses of the DC cancer vaccine of the invention to the subject, wherein the subject is one whom has previously been administered a therapeutic dose of the DC cancer vaccine of the invention. Each of the one or more subsequent therapeutic doses are a dose sufficient to elicit an immune response against a cancer cell in the subject (i.e. a primary immune response and/or a secondary immune response).

In embodiments comprising administering one or more subsequent therapeutic doses of the DC cancer vaccine of the invention to the subject, preferably 2, 3, 4, 5, 6, 7, 8, 9, 10 or n subsequent therapeutic doses of the DC cancer vaccine are administered to the subject; wherein “n” is any number of doses greater than 10 doses (for example 11, 12, 13, 14, 15, 16, 17, 18, 19, 20, 21, 22, 23, 24, 25, 26, 27, 28, 29 or 30 doses). Preferably, the number and frequency of subsequent therapeutic doses administered to the subject is sufficient to treat or prevent cancer in the subject.

In preferred embodiments, the DC cancer vaccine administered to the subject as a subsequent therapeutic dose has been exposed to the same type(s) of antigenic construct and/or phagocytosable particles as the DC cancer vaccine of the invention previously administered to the subject as a therapeutic dose.

In embodiments comprising administering one or more subsequent therapeutic doses of the DC cancer vaccine of the invention to the subject, preferably, the one or more subsequent therapeutic doses are administered to the subject at intervals of days, weeks or months. For example, one or more subsequent therapeutic doses are administered to the subject every day, every second day, every third day, every fourth day, every fifth day, every sixth day. Alternatively, or additionally, one or more subsequent therapeutic doses are administered to the subject, for example, once every week, once every two weeks, once every three weeks or once every four weeks. Alternatively, or additionally, one or more subsequent therapeutic doses are administered to the subject, for example, once every month, once every two months, once every three months or once every 6th month. Alternatively, or additionally, one or more subsequent therapeutic doses are administered to the subject, for example, once every year.

In certain preferred embodiments, the use of the DC cancer vaccine for the treatment or prophylaxis of cancer comprises administering one or more therapeutic doses of the DC cancer vaccine to the subject, wherein the subject is one whom has previously been, or is simultaneously being, administered a dose of anticancer T-cells, preferably a therapeutic dose of anticancer T-cells. In such embodiments, preferably the dose of anticancer T-cells administered to the subject contains at least one anticancer T-cell that recognises an epitope presented on the surface of a DC contained in the DC cancer vaccine administered to the subject.

For example, in one exemplary embodiment, the use of the DC cancer vaccine for the treatment or prophylaxis of cancer comprises administering to a subject a dose of anticancer T-cells followed by administering a dose of a DC cancer vaccine of the present invention three weeks after administration of the dose of anticancer T-cells, and one or more subsequent doses of a DC cancer vaccine once a week thereafter (for example, administering a dose of anticancer T-cells during week 0, followed by a first dose of a DC cancer vaccine of the invention during week 3, a second dose of the DC cancer vaccine of the invention during week 4, a third dose of the DC cancer vaccine of the invention during week 5, and so on, until the end of the treatment cycle. In such an exemplary embodiment, the one or more doses of DC cancer vaccine administered to the subject preferably contains at least one DC presenting an epitope recognised by the previously administered dose of anticancer T-cells.

Administering a DC cancer vaccine that contains at least one DC presenting an epitope recognised by a previously administered dose of anticancer T-cells, acts to boost the immune response of the previously administered dose of anticancer T-cells. One or more doses of the DC cancer vaccine of the invention therefore prolongs the anticancer effects of the previously administered dose of anticancer T-cells.

Ex Vivo Expansion of Anticancer T-Cells from Blood

The DC cancer vaccine of the invention finds use in the ex vivo activation and expansion of anticancer T-cells, said use comprising the steps of:
a) harvesting anticancer T-cells, and optionally antigen-presenting cells (APCs), from a blood sample from the subject;
b) expanding the anticancer T-cells in vitro; and
c) administering a therapeutic dose of the expanded anticancer T-cells to the subject.

In certain embodiments, steps a), b) and c) are performed before administering the DC cancer vaccine of the invention to the subject. In certain other embodiments, steps a), b) and c) are performed after administering the DC cancer vaccine of the invention to the subject. In certain other embodiments, steps a), b) and c) are performed before and after administering the DC cancer vaccine of the invention to the subject.

Administration of the DC cancer vaccine results in the in vivo activation and expansion of anticancer T-cells in a subject. As such, following administration of the DC cancer vaccine, the levels of circulating anticancer T-cells may increase compared to the levels prior to dosing with the DC cancer vaccine.

Thus, in embodiments wherein steps a), b) and c) are performed before administering the DC cancer vaccine of the invention to the subject, the DC cancer vaccine activates and expands the pre-administered anticancer T-cells within the subject therefore enhancing the anticancer effects of the therapeutic dose of the expanded anticancer T-cells administered to the subject in step c). In embodiments wherein steps a), b) and c) are performed after administering the DC cancer vaccine of the invention to the subject, the prior administration of the DC cancer vaccine increases the level of circulating anticancer T-cells, thus providing a readily accessible source of anticancer T-cells that can be isolated from the subject, expanded ex vivo, and used as an anticancer T-cell therapy.

Step a): Harvesting Anticancer T-Cells from the Subject

In step a), anticancer T-cells are harvested from a subject following administration of a dose or several doses (preferably therapeutic doses) of a DC cancer vaccine of the present invention to the subject. Alternatively, or additionally, anticancer T-cells may be harvested from a subject at the same time and/or before administrating a dose (preferably a therapeutic dose) of a DC cancer vaccine of the present invention to the subject. Optionally, APCs may be harvested from a subject simultaneously, sequentially or separately to the harvesting of anticancer T-cells from the subject. Preferably, the APCs are harvested at the same time as harvesting the anticancer T-cells from the subject. Preferably, the APCs are derived from the same sample as the anticancer T-cells. For example, the APC and anticancer T-cells may both be derived from a PBMC sample from the subject. Preferably, the APC is a monocyte, DC, B-cell or macrophage, or other cell that can either phagocytose or internalise extracellular molecules, such as antigens, and present antigen-derived peptides on MHC class II and/or MHC class I molecules to CD4+ T-cells and/or CD8+ T cells. More preferably, the APC is a DC.

The anticancer T-cells are harvested from a blood sample derived from a subject. Preferably the blood sample is a PBMC sample. The PBMC sample may be freshly used or it may be subjected to freezing for storage before use. The anticancer T-cells may also be derived from lymphocytes isolated by leukapheresis.

It is not necessary to do so, but in some circumstances the anticancer T-cells may also be derived from a tumour of the subject, from the tumour itself (i.e. Tumour Infiltrating Lymphocytes, TILs), for example from a metastatic tissue sample or a primary tumour tissue sample, or a sample derived from a lymphatic vessel in a tumour or a tumour draining lymph node (i.e. a sentinel node) or from any lymph node derived from the subject.

Step b): In Vitro Expansion of Anticancer T-Cells

The anticancer T-cells harvested from the subject in step a) may be used in the preparation of T-cell samples enriched for anticancer T-cells suitable for use in the treatment or prophylaxis of cancer in the subject. Anticancer T-cells suitable for administration to the subject are prepared by in vitro activation and expansion of anticancer T-cells harvested from the subject. The in vitro activation and expansion method comprises the steps of:
ba) optionally providing a phagocytosable particle phagocytosable particle comprising a core and an antigenic construct tightly associated to the core, wherein the antigenic construct comprises a epitope peptide having an amino acid sequence corresponding to an amino acid sequence of a part of a protein or peptide known or suspected to be expressed by a cancer cell in the subject;
bb) providing an APC;
bc) optionally contacting the phagocytosable particle with the APC from step bb) in vitro and under conditions allowing phagocytosis of the phagocytosable particle by the APC;
bd) providing an anticancer T-cell sample harvested from a blood sample from the subject; and
be) contacting the anticancer T-cell sample harvested from the subject with the APC from step bb) or bc) in vitro and under conditions allowing for specific activation and expansion of anticancer T-cells in response to antigen presented by the APC.

For the avoidance of doubt, a phagocytosable particle provided in step ba) may independently have any of the properties and/or characteristics of the phagocytosable particles described herein and used in the method of manufacturing a DC cancer vaccine according to the present invention.

In embodiments, the in vitro expansion method may comprise contacting the APC with 100 to 1×10⁹ phagocytosable particles, for example 100 to 1×10⁸ phagocytosable particles, for example 100 to 1×10⁷ phagocytosable particles, or for example 1000 to 1×10⁷ phagocytosable particles. The ratio of phagocytosable particle to APC may be in the range 1000:1 to 1:10. The ratio can be optimised depending on the size of the phagocytosable particle. For example, for a phagocytosable particle with a largest diameter of about 1 μm, the ratio may be in the range 50:1 to 2:1, for example 25:1 to 5:1, 15:1 to 7:1, and 10:1.

In certain embodiments, an APC is contacted with a phagocytosable particle that comprises the same type(s) of antigenic construct as a phagocytosable particle used in the method of manufacturing a DC cancer vaccine according to the present invention.

In preferred embodiments, the APC provided in step bb) is a DC from the DC cancer vaccine of the invention, and step be) comprises contacting the anticancer T-cell sample with the DC from step bb). Preferably, the DC is from a DC cancer vaccine of the invention that has previously been administered to the subject. For example, the DC may be from the same batch of DC cancer vaccine administered (or planned to be administered) to the subject following step c) of the method, or from the same batch of DC cancer vaccine of the invention previously administered to the subject. Or, the DC may be from a DC cancer vaccine of the invention prepared using the same method as a DC cancer vaccine administered (or planned to be administered) to the subject following step c) of the method, or from a DC cancer vaccine of the invention prepared using the same method as a DC cancer vaccine previously administered to the subject. Or, the DC may be from a DC cancer vaccine of the invention prepared using phagocytosable particles from the same group as those used to prepare a DC cancer vaccine administered (or planned to be administered) to the subject following step c) of the method (i.e. the phagocytosable particles have the same core and the same type of antigenic constructs tightly associated to the core), or from a DC cancer vaccine of the invention prepared using phagocytosable particles from the same group as those used to prepare the DC cancer vaccine previously administered to the subject (i.e. the phagocytosable particles have the same core and the same type of antigenic constructs tightly associated to the core).

In one embodiment, the method of expanding anticancer T-cells in vitro comprises adding low doses of IL2 to the anticancer T-cell sample, for example greater than 1.25 U/ml (for example 1.25 U/ml, 2.5 U/ml, 5 U/ml, or 50 U/ml), preferably greater than 2.5 U/ml, 5 U/ml, or 50 U/ml of IL2 to the anticancer T-cell sample. Antigen specific T-cell expansion occurs in the presence of the antigen-presenting cell when IL2 is simultaneously present. The IL2 promotes the differentiation of anticancer T-cells into effector anticancer T-cells and into memory anticancer T-cells. Following expansion of anticancer T-cells, the APCs may be removed from the expanded T-cell population, for example by magnetic separation.

In another embodiment, the method of expanding anticancer T-cells in vitro comprises adding IL2 and/or IL7 and/or IL15 to the anticancer T-cell sample, for example a low dose of IL2 to the anticancer T-cell sample, for example greater than 1.25 U/ml (for example 1.25 U/ml, 2.5 U/ml, 5 U/ml, or 50 U/ml), preferably greater than 2.5 U/ml, 5 U/ml, or 50 U/ml of IL2, with optional addition of IL7 and/or IL15. For example a low dose of IL7 to the anticancer T-cell sample, for example greater than 1.25 U/ml (for example 1.25 U/ml, 2.5 U/ml, 5 U/ml, or 50 U/ml), preferably greater than 2.5 U/ml, 5 U/ml, or 50 U/ml of IL7; and/or for example, a low dose of IL15 to the anticancer T-cell sample, for example greater than 1.25 U/ml (for example 1.25 U/ml, 2.5 U/ml, 5 U/ml, or 50 U/ml), preferably greater than 2.5 U/ml, 5 U/ml, or 50 U/ml of IL15.

In preferred embodiments, the method of activating and expanding anticancer T-cells in vitro comprises a step of removing the APCs that contain a phagocytosable particle from the anticancer T-cells. In embodiments where the phagocytosable particles comprise a magnetic core, the APCs are removed from the anticancer T-cells by using magnetic separation.

Following contact of an anticancer T-cell with an APC that contains a phagocytosable particle described herein, the degree of anticancer T-cell activation may be determined, for example by comparing the degree of anticancer T-cell activation to a relevant reference. Determining the degree of anticancer T-cell activation may be performed using T-cell activation assays known in the art, for example an ELISpot, FluoroSpot, intracellular staining of cytokines with flow cytometry, FASCIA (Flow-cytometric Assay for Specific Cell-mediated Immune-response in Activated whole blood), proliferation assays (e.g. thymidine incorporation, CFSE or BrdU staining), specific TCR-detection with MHC-I or II tetramers, and ELISA- or Luminex analysis of secreted cytokines ELISpot assays. The method may comprise the step of comparing the degree of anticancer T-cell activation to a relevant reference. Suitable references include, for example, a T-cell sample that does not comprising anticancer T-cells, or an anticancer T-cell sample that has not been contacted with an APC that contains a phagocytosable particle described herein.

c) Administering a Therapeutic Dose of the Expanded Anticancer T-Cells to the Subject.

A therapeutic dose of the expanded anticancer T-cells may be administered to the subject. Thus, the present invention also provides anticancer T-cells for use in treating or preventing cancer in a subject. The expanded anticancer T-cells may be administered to the subject intravenously, intraarterially, intrathecally or intraperitoneally. In certain embodiments, the subject whom receives a dose of the anticancer T-cells is one whom has previous been administered a DC cancer vaccine of the invention.

The precise dosage of the expanded anticancer T-cells will vary with the dosing schedule, the age, size, sex and condition of the subject (typically mammal or human), the nature and severity of the condition, and other relevant medical and physical factors. Thus, a precise therapeutically effective amount can be readily determined by the clinician, for example, by performing a dose escalation trial where escalating doses of the anticancer T-cells are administered to a subject. For humans, an effective dose will be known or otherwise able to be determined by one of ordinary skill in the art.

Anticancer T-cells expanded using the method of the present invention may be administered to a subject with cancer. In preferred embodiments, the dose of expanded anticancer T-cells is administered to a subject, wherein the subject is one from whom the anticancer T-cells were harvested.

The dose of expanded anticancer T-cells may be formulated in any pharmaceutically acceptable formulation. Such formulations are known in the art and include, for example, aqueous sterile injectable solutions (e.g. saline, such as PBS), cell culture mediums suitable for clinical use and autologous plasma, which may also contain anti-oxidants, buffering agent (e.g. sodium phosphate, potassium phosphate, TRIS and TEA), bacteriostats, and solutes which render the T-cell population dose isotonic with the blood of the intended recipient.

The dose of expanded anticancer T-cells preferably comprises CD4+ T-cells and/or CD8+ T-cells, for example a mixture of CD4+ T-cells and CD8+ T-cells. For example, in embodiments, the dose of anticancer T-cells may predominantly comprise CD4+ T-cells and/or CD8+ T-cells. In embodiments, the dose of expanded anticancer T-cells predominantly comprises T-cells that specifically bind to at least one epitope having an amino acid sequence corresponding to an amino acid sequence of a part of a protein or peptide known or suspected to be expressed by a cancer cell in a subject.

Additionally or alternatively, a dose of expanded anticancer T-cells may be simultaneously, sequentially or separately administered with a cytokine, for example IL2, IL15 or other cytokines which can drive the survival and proliferation of the anticancer T cells. Typically, the administration of the cytokines is continued after the administration of the anticancer T-cells to prolong the circulation and survival of the expanded anticancer T-cell following administration to the subject.

### EXAMPLES

### Example 1: General Protocol for Coupling Antigenic Constructs to Magnetic Cores

Coupling of Antigenic Constructs or Model Peptides/Proteins to a Core:

Dynabeads® MyOne™ Carboxylic Acid (ThermoFischer Scientific) were used (1 μm diameter spheres) as the core. Dynabeads® MyOne™ Carboxylic Acid particles are paramagnetic polystyrene particles comprising iron oxide and functionalised on the surface of the particle with free carboxylic acid groups. The coupling procedure was carried out according to the manufacturer's protocol (Two-Step procedure using NHS (N-Hydroxysuccinimide) and EDC (ethyl carbodiimide)):

Step 1): The polystyrene particles are washed twice with MES-Buffer (25 mM MES (2-(N-morpholino)ethanesulfonic acid), pH 6). The carboxylic acid groups were then activated by adding 50 mg/ml NHS (N-Hydroxysuccinimide) and 50 mg/ml EDC (N-(3-Dimethylaminopropyl)-N′-ethylcarbodiimide) in MES-buffer to the polystyrene particles and incubated for 30 min at room temperature (RT). The polystyrene particles were collected with a magnet and the supernatant was removed and the polystyrene particles washed twice with MES-buffer.

Step 2): The neoantigenic construct or model peptide/protein sample was diluted in MES-buffer to a concentration of 1 mg/ml, total 100 μg and added to the polystyrene particles and incubated for 1 h at RT. The polystyrene particles were collected with a magnet and the supernatant was removed and saved for peptide-concentration measurement. The non-reacted activated carboxylic acid groups were quenched with 50 mM Tris pH 7.4 for 15 min. The polystyrene particles were then washed with PBS pH 7.4 and then stored in −80° C.

A BCA (bicinchoninic acid) protein assay kit (Pierce BCA Protein Assay Kit, ThermoFisher Scientific) was used according to the manufacturer's protocol, to measure the amount of peptidic material coupled to the polystyrene particles and to measure the peptidic material concentration of the neoantigenic construct sample before coupling as well as the peptidic material concentration of the supernatant after coupling.

Several polypeptides were tested and an estimated average of 48.7 μg (mean: 48.7, SD: 20.5, N=10) neoantigenic construct was coupled per 1 mg polystyrene particles. According to manufacturer's instruction, 50 μg polypeptide can be coupled per 1 mg particles, indicating that the efficiency of the coupling achieved was high.

### Example 2: Washes

Polystyrene particles were coupled to recombinant neoantigenic constructs produced in E. coli according to the method described in Example 1. After coupling, the polystyrene particles were washed with one of 3 different wash-buffers: 2M NaOH pH 14.3, 8 M Urea or 6 M Guanidine (Guanidine-HCl), all in sterile water at RT, or they were incubated in PBS at 95° C. The polystyrene particles were suspended in the buffer and shaken for 4 min, collected with a magnet and the supernatant removed. This was repeated 3 times. The heat treated polystyrene particles were put in PBS pH 7.4 and put in a heating block at 95° C. for 5 minutes, then collected with a magnet and the supernatant removed. This was repeated 3 times. The particles were then washed 3 times with sterile PBS to remove any remaining wash-buffer.

Four different washing conditions were tested: (a) High pH (2M NaOH pH 14.3), (b) Heat (95° C.) and sterilising/denaturing agents ((c) 8M Urea and (d) 6M guanidine hydrochloride). In every case, the neoantigenic constructs associated with the polystyrene particles remained associated with the polystyrene particles.

### Example 3: Identification of Suitable Particle Size for Phagocytosable Particles

A cell proliferation assay measuring Thymidine incorporation was used to test the effect of phagocytosable particle size on antigen-specific T-cell activation. Splenocytes from ovalbumin (OVA) immunized mice were stimulated with OVA coupled polystyrene particles of different sizes to measure antigen specific proliferation.

Dynabeads® MyOne™ Carboxylic Acid particles with a diameter of 5.6 μm, 1 μm and 0.2 μm were coupled with OVA (OVA-particles) or bovine serum albumin (BSA-particles) according to the protocol in Example 1.

To test the effectiveness of the OVA-particles to stimulate antigen specific T-cell activation a proliferation assay (with 3H thymidine incorporation) was used. Particle concentration in relation to cell concentration was 1:1 for the 5.6 μm particles, 10:1 for the 1 μm particles and 500:1 for the 0.2 μm particles. Total protein concentration during the incubation with the cells was calculated to 125 ng/ml, 160 ng/ml and 160 ng/ml for the 5.6 μm, 1 μm and 0.2 μm respectively. The proliferation assay was run as follows:

As stimuli, ovalbumin (SigmaAldrich) and BSA (SigmaAldrich) coupled to Dynabeads® MyOne™ Carboxylic Acid particles were used (OVA-particles or BSA-particles). Mice were immunized to ovalbumin via monthly injections of 100 μg ovalbumin (Sigma) adsorbed to aluminium hydroxide. Three months after the first injection the mice were killed and spleens harvested. Splenocytes were prepared by standard procedures, as described in Thunberg et al. 2009, Allergy 64:919.

The cells were incubated in cRPMI either with OVA-particles or BSA-particles (10 particles per cell) for 5 days. All cells were incubated for 6 days in a humidified atmosphere with 6% CO₂ at 37° C. One μCu/well [³H] thymidine was added to cell cultures for the final 18 h of incubation. Mean counts per minute (cpm) obtained from stimulated triplicates were divided by mean cpm values from un-stimulated cells and expressed as stimulation indices (SI). SI-values≥2.0 are generally considered positive.

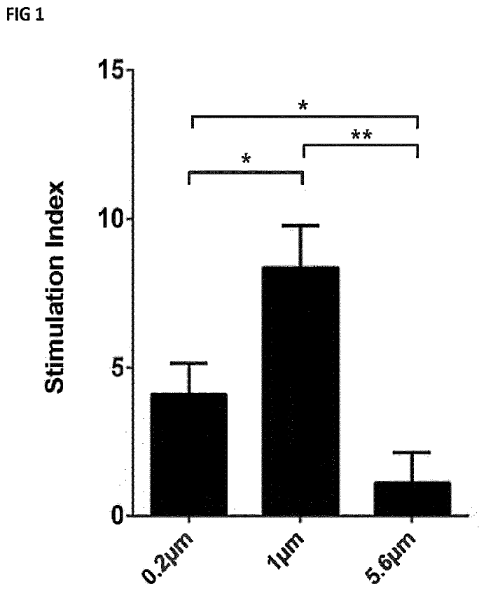

As seen in FIG. 1, cells incubated with OVA-particles with a diameter of 0.2 μm showed increase in proliferation with a mean SI of 4.1 (95% CI 2.4-5.8, P=0.007). The cells incubated with OVA-particles with a diameter of 1 μm showed increase in proliferation with a mean SI of 8.4 (95% CI 6.1-10.6, P<0.005). The cells incubated with OVA-particles with a diameter of 5.6 μm failed to stimulate proliferation, mean SI 1.1 (95% CI 0.4-2.7, P=0.876).

These results show that antigen coupled to particles of different sizes can stimulate cell proliferation. The particles with a diameter of about 1 μm seems to be most efficient in regards to cell stimulation but particles down to a size of 0.2 μm still works. It is reasonable to predict that particles of sizes larger than 1 μm also work, although as the diameter comes close to 5.6 μm the particles completely fail to stimulate the cells. It is reasonable to assume that 1 μm is an optimal size, since it is similar to the size of bacteria. Our immune system has evolved to phagocytose and react to microorganisms/particles of this size. A normal antigen presenting cell has a size in the range 10-15 μm.

### Example 4: Comparison of Antigen Coupled Particles of Different Particle Sizes and their Effectiveness in Activating and Expanding T-Cells

(i) Preparation of Antigen Coupled Phagocytosable Particles:

Three kinds of paramagnetic polystyrene phagocytosable particles of different sizes were used:
diameter of 1 μm (Dynabeads MyOne Carboxylic Acid, ThermoFisher),
diameter of 2.8 μm (Dynabeads M-270 Carboxylic acid, ThermoFisher) and
diameter of 4.5 μm (Dynabeads M-450 Epoxy, ThermoFisher).

The phagocytosable particles were coupled with the model antigen Cytomegalovirus (CMV) protein pp65 construct (SEQ ID NO: 1) according to the manufacturer's instruction. To remove endotoxin, the phagocytosable particles were washed five times with a 0.75M sodium hydroxide buffer and subsequently resuspended in sterile PBS.

(ii) Incubation of Antigen Coupled Phagocytosable Particles:

Peripheral blood mononuclear cells (PBMCs) from a CMV-sensitive healthy donor, isolated via standard ficoll-based density gradient centrifugation were cultured together with the phagocytosable particles coupled with the CMV construct (hereinafter referred to as “CMV-particles”) in a 48-well plate for 18 h at 37° C., 5% CO₂, 500,000 cells/well at a concentration of 1,000,000 cells/ml. The concentration of CMV-particles were equalized based on total surface area (a surrogate marker for CMV amount as it is bound to the surface of the CMV-particles). This equalled to 10 CMV-particles/PBMC for the 1 μm sized particles, 1.4 CMV-particle/PBMC for the 2.8 μm sized particles and 0.5 CMV-particles/PBMC for the 4.5 μm sized particles, based on the number of total PMBCs in the sample. The results for each particle sized is shown below in Table 1.

TABLE 1

| Particle | | Number of | Ratio of CMV- | Number of CMV- |
|---|---|---|---|---|
| size | | PBMCs in sample | particles to PBMCs | particles in sample |
| 1 | μm | 500,000 | 10:1 | 5,000,000 |
| 2.8 | μm | 500,000 | 1.4:1 | 700,000 |
| 4.5 | μm | 500,000 | 0.5:1 | 250,000 |

(iii) Assessment of Uptake:

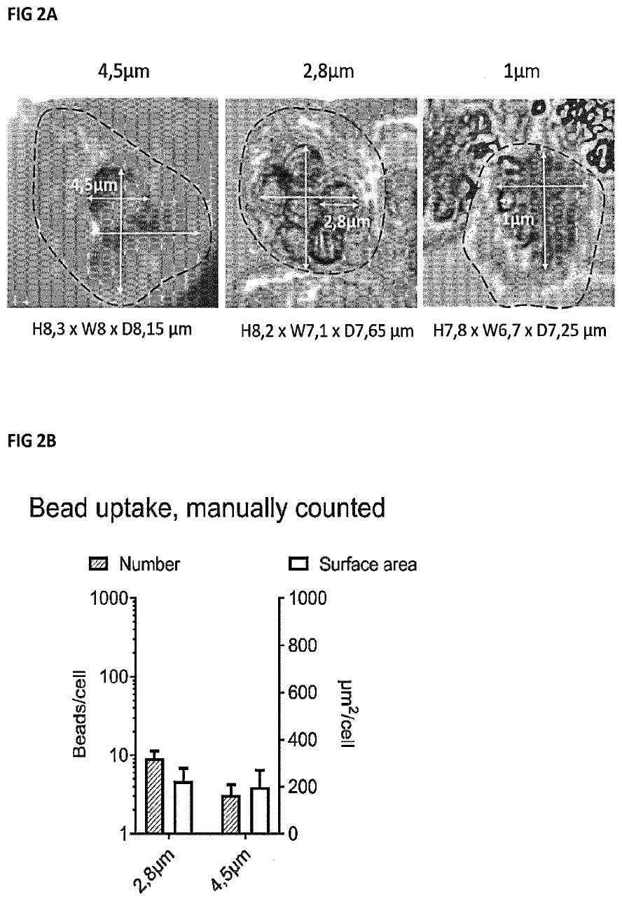

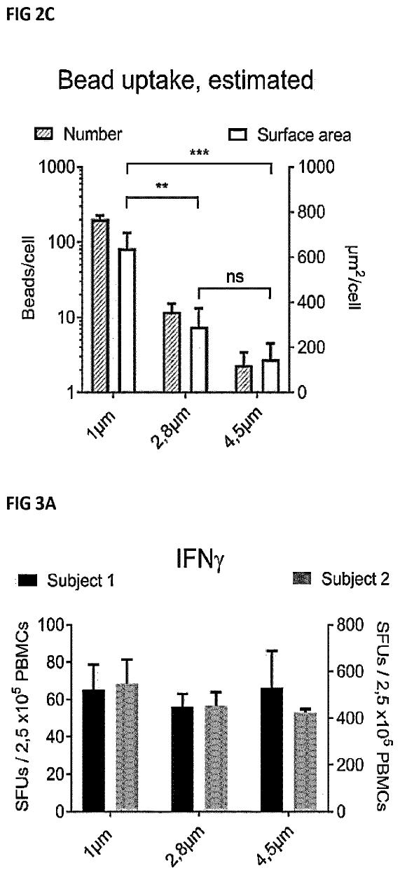

After incubation, the number of phagocytosed CMV-particles were manually counted using a confocal microscope. Eight cells were counted to obtain mean and standard deviation values. In FIG. 2A, there are shown images from the confocal microscope of representative cells with intracellular phagocytosed CMV-particles. Black dashed line indicates the outline of the cell. The white line shows the dimensions of the total intracellular CMV-particles.

This method was not applicable for the 1 μm CMV-particles as they were too small to accurately count. In order to assess the amount of 1 μm CMV-particles, the total volume of all phagocytosed CMV-particles were measured and the amount of individual CMV-particles were back-calculated based on the total volume, assuming a packing density of 60%. This method proved reasonably accurate for the 2.8 μm CMV-particles (manually counted 9.1 CMV-particles/cell vs estimated 11.9 CMV-particles/cell) and 4.5 μm CMV-particles (manually counted 3.1 CMV-particles/cell vs estimated 2.4 CMV-particles/cell) and can as such be assumed to accurately estimate the amount of 1 μm CMV-particles as well.

The uptake of CMV-particles in shown in FIGS. 2B and 2C. FIG. 2B shows the number of CMV-particles taken up by each cell as assessed by manual counting (8 cells counted per bead-type). Using the manual counting method, it was found that the number of phagocytosed particles per cell for the 4.5 μm CMV-particles was of 3.1 (±1.1). For the 2.8 μm CMV-particles it was 9.1 (±2.2). It was not possible to count the number of 1 μm CMV-particles using this method.

FIG. 2C shows the number of CMV-particles taken up by each cell as assessed by the volume calculation (3 cells measured per bead-type) (*p<0.05 **p<0.01 ***p<0.001, calculated using Students T-test). Using the volume calculation method, it was found that the number of phagocytosed particles per cell for the 4.5 μm CMV-particles was of 2.4 (±1.1). For the 2.8 μm CMV-particles it was 11.9 (±3.2). For the 1 μm CMV-particles it was 203.7 (±21.9).

Based on the number of CMV-particles taken up by each cell as assessed by the volume calculation method, the total phagocytized surface area, and by extension the total amount of CMV, was calculated. The surface area that was taken up was calculated as 639.6 (±68.9) μm² for the 1 μm CMV-particles, 293.1 (±79.3) μm² for the 2.8 μm CMV-particles and 150.7 (±67.0) μm² for the 4.5 μm CMV-particles. These data are shown in Table 2 below.

TABLE 2

| | | | CMV-particles | | |
|---|---|---|---|---|---|
| | | CMV-particles | uptake per cell | | Surface area |
| Particle | | uptake per cell | (calculated | | taken up |
| size | | (counted) | from volume) | | per cell |
| 1 | μm | — | 203.7 | (±21.9) | 639.6 (±68.9) μm² |
| 2.8 | μm | 9.1 (±2.2) | 11.9 | (±3.2) | 293.1 (±79.3) μm² |
| 4.5 | μm | 3.1 (±1.1) | 2.4 | (±1.1) | 150.7 (±67.0) μm² |

(iv) Assessment of T-Cell Stimulation

The ability of the antigen coupled particles to stimulate T-cells and hence promote their expansion was assessed by measuring the release of IFNγ, IL22 and IL17A from PBMCs using a FluoroSpot assay (Mabtech, Sweden). PBMCs (250,000/well) from CMV-sensitive healthy donors (n=2) were stimulated with the CMV-particles in triplicates. The concentration of antigen coupled particles were as previously described equalized based on total surface area: 10×1 μm CMV-particles/cell, 1.4×2.8 μm CMV-particles/PBMC and 0.5×4.5 μm CMV-particles/cell. The number of PBMCs per well of the FluoroSpot assay is shown below (Table 3) for each particle size, together with the estimate number of monocytes per well (based on an estimated 20% monocyte content of a PBMC sample).

TABLE 3

| particle | | | |
|---|---|---|---|
| size | | Number of PBMCs per well | Number of monocytes per well |
| 1 | μm | 250,000 | 50,000 |
| 2.8 | μm | 250,000 | 50,000 |
| 4.5 | μm | 250,000 | 50,000 |

The PBMCs were incubated for 44 h at 37° C., 5% CO₂. The plates were developed according to the manufacturer's instructions and read in an automated FluoroSpot reader. The data reported for the FluoroSpot is spot-numbers when the cells are stimulated with CMV-particles above the spot-numbers when not stimulated with CMV-particles.

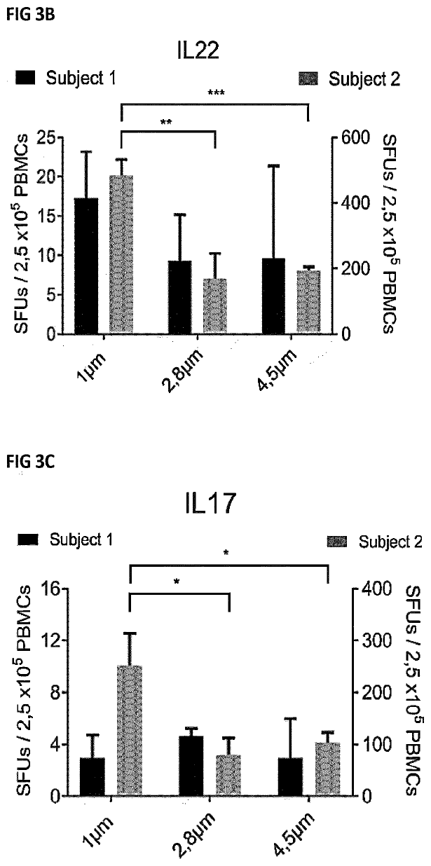

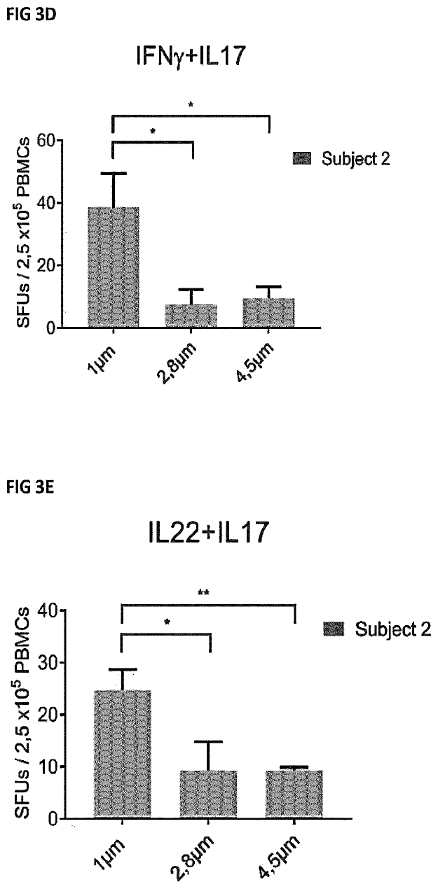

The level of IFNγ-production, as assessed in the FluoroSpot assay, is shown in FIG. 3A. It is seen that there is little difference between the CMV-particles.

The level of IL22 and IL17 production, as assessed in the FluoroSpot assay, is shown in FIGS. 3B and 3C. It is seen that the 1 μm CMV-particles caused a significantly higher IL22 and IL17 production in one individual than the larger CMV-particles, with a similar trend seen for the other individual in regards to IL22.

The level of dual-cytokine production, as assessed in the FluoroSpot assay, is shown in FIGS. 3D and 3E. It is seen that the 1 μm CMV-particles caused a significantly higher dual-cytokine release (IFNγ+IL17 and IL22γ+IL17) for one healthy donor when stimulated with the 1 μm antigen coupled particles than with the larger CMV-particles.

The cytokine release in these experiments serves as a proxy for T-cell expansion. In general, IFNγ is produced by CD4+ T-cells (Th1 subclass) and CD8+ T-cells. IL17 and IL22 are mainly produced by pro-inflammatory Th17 CD4+ T-cells. Such cells are pro-inflammatory have been shown to assist in tumour eradication. The data suggest that the 1 μm beads activate and cause expansion of Th1 CD4+ T-cells and CD8+ T-cells to the same degree as the other beads, with the added benefit of also activating and causing expansion of additional pro-inflammatory Th17 CD4+ T-cells and the less distinct but still pro-inflammatory double cytokine producing T-cells.

### Example 5: Exemplification of Phagocytosable Particle Sterilisation Protocol Using Bacillus subtilis

This experiment was performed under sterile conditions in a laminar air flow (LAF) safety cabinet. Phagocytosable particles comprising a core (Sera-Mag SpeedBeads Carboxylate-Modified magnetic particles, Cytiva) attached to a neoantigen construct were washed four times with high concentration alkaline solution (2M to 5M NaOH). After the first wash, the phagocytosable particles were transferred to a new sterile tube, the supernatant was removed, and a second volume of alkaline solution was added. Phagocytosable particles were sonicated for 10 minutes in a sonication bath, and then incubated for 30 minutes with end-over-end rotation in the same alkali solution. This process was repeated a further two times, followed by four washes with sterile Dulbecco-modifies PBS.

Effectiveness of the NaOH treatment protocol was evaluated by spiking phagocytosable particles (Sera-Mag SpeedBeads Carboxylate-Modified magnetic particles, without attached neoantigen) with a high load (>1.2 CFU) of Bacillus subtilis subsp. spizizenii (ATCC® 6633™ Epower 106 CFU), followed by the above described NaOH treatment protocol. After NaOH treatment, both full bead suspension and supernatant from non-treated (positive control) vs 5M or 2M NaOH treated samples were plated on nutrient agar in the absence of antibiotics, and incubated at 37° C. overnight (>16 hours).

Results

Both 5M and 2M NaOH treatment effectively abolished bacterial growth, whereas colonies of Bacillus subtilis subsp. spizizenii were abundantly growing in the absence of washes. In conclusion, both the 2M and 5 M NaOH treatments were highly effective at removing an artificially high bioburden load from the phagocytosable particles.

### Example 6: Preparation of a DC Cancer Vaccine Using Human DCs

Dendritic cell cancer vaccines were prepared using phagocytosable particles comprising a neoantigenic construct containing six neoepitope peptides identified in HLA-A*02.01-positive melanoma tumor cells resected from a male patient with stage III auxiliary lymph node metastatic melanoma (hereinafter referred to as patient ANRU). Two different neoantigenic constructs were used in this example: neoantigenic construct #1 (SEQ ID NO: 3) and neoantigenic construct #2 (SEQ ID NO: 4). Further details of the neoantigenic construct design are shown in FIGS. 9A and 9B.

The phagocytosable particles used in this example which comprise neoantigenic construct #1 are hereinafter referred to as Example particle #1, and the phagocytosable particles used in this example which comprise neoantigenic construct #2 are hereinafter referred to as Example particle #2.

To assess the ability of the DC vaccines prepared using Example particles #1 or #2 to activate anticancer T-cells, a set of comparative DC vaccines were prepared using control particles attached to wild-type antigenic constructs #1 or #2, or prepared using soluble peptides (NUP210 wild-type, NUP210 neoantigen, ETV6 wild-type, or ETV6 neoantigen), or tumor cell lysates prepared from tumor cells obtained from patient ANRU. For the avoidance of doubt, wild-type antigenic constructs #1 or #2 are wild-type (i.e. non-mutated) versions of neoantigenic constructs #1 and #2, respectively. The phagocytosable particles coupled to wild-type antigenic constructs #1 or #2 (SEQ ID NOs: 5 and 6) are herein referred to as Control particles #1 and #2, respectively.

The peptide sequences of each neoantigenic construct, wild-type antigenic construct and soluble peptide used in this example are shown in Table 4.

TABLE 4
Peptide sequences

| construct/peptide | Peptide sequence |
|---|---|
| Neoantigenic | MKAIGGSKGGSKGGSKGSWGSKGGSKGGSKGGSKGGSAVPLQSLTGEV |
| construct #1 (SEQ ID | FAHASLFVHVAGGSATSQFKGYRRKSSLNGKGESSGGSAIYEVPKEMHE |
| NO: 3) | NKQHLQKDFFGGSADREFQFFDHNLMESIKMGDPGGSGQTAIDAALTF |
| | VVDQDGGVHIGGSMPIGRIADCRVLWDYVYQLLSSGHHHHHHHH |
| Neoantigenic | MKAIGGSKGGSKGGSKGSWGSKGGSKGGSKGGSKGGSAVPLQSLTGEV |
| construct #2 | FAHASLFVHVAGGSATSQFKGYRRKSSLNGKGESSGGSAIYEVPKEMHE |
| (SEQ ID NO: 4) | NKQHLQKDFFGGSADREFQFFDHNLMESIKMGDPGGSASPLKSIWSVL |
| | TPSPIKSTLGGGSMPIGRIADCRVLWDYVYQLLSSGHHHHHHHH |
| Wild-type antigenic | MKAIGGSKGGSKGGSKGSWGSKGGSKGGSKGGSKGGSAVPLQSLTGEV |
| construct #1 | LAHASLFVHVAGGSATSQFKGYRRRSSLNGKGESSGGSAIYEVPKEMHG |
| (SEQ ID NO: 5) | NKQHLQKDFFGGSADREFQFFDHHLMESIKMGDPGGSGQTAIDAALTS |
| | VVDQDGGVHIGGSMPIGRIADCRLLWDYVYQLLSSGHHHHHHHH |
| Wild-type antigenic | MKAIGGSKGGSKGGSKGSWGSKGGSKGGSKGGSKGGSAVPLQSLTGEV |
| construct #2 | LAHASLFVHVAGGSATSQFKGYRRRSSLNGKGESSGGSAIYEVPKEMHG |
| (SEQ ID NO: 6) | NKQHLQKDFFGGSADREFQFFDHHLMESIKMGDPGGSASPLKSIWSVS |
| | TPSPIKSTLGGGSMPIGRIADCRLLWDYVYQLLSSGHHHHHHHH |
| ETV6 neoantigen | VLWDYVYQL |
| (SEQ ID NO: 7) | |
| NUP210 neoantigen | AIDAALTFV |
| (SEQ ID NO: 8) | |
| NUP210 wild-type | AIDAALTSV |
| (SEQ ID NO: 9) | |
| ETV6 wild-type | LLWDYVYQL |
| (SEQ ID NO: 10) | |

The core of the phagocytosable particles used in this example are magnetic polystyrene particles (Sera-Mag SpeedBeads (hydrophilic) Carboxylate-Modified Magnetic particles, Cytiva). Each phagocytosable particle was prepared and washed in line with the method described in Examples 1 and 2.

For the avoidance of doubt, the soluble peptides and tumor cell lysates used in this example were not associated to a core and therefore not used in the form of a phagocytosable particle as described herein.

a) DC Vaccine Preparation Method Using Healthy Donor Blood:

Healthy donor blood was processed using Ficoll-Hypaque (Cytiva) density gradient centrifugation according to the manufacture's guidelines to provide a PBMC sample. Monocytes and CD8⁺ T-cells were isolated from the PBMC sample by magnetic cell isolation using MACS® CD14 or CD8 beads (Miltenyi Biotec), respectively, according to the manufacturer's guidelines. The isolated CD8+ T-cells were frozen until required for use as described in section c) below.

The isolated monocytes were matured into immature DC (iDC) following the method described by Wickstrom et al (Front Immunol. 2019; 10: 2766). In brief, monocytes were cultured at 2×10⁶ cells/ml in CellGro® media supplemented with 20 ng/ml IL-4 and 100 ng/mL GM-CSF for 48 h to mature into iDCs. iDC were harvested using a cell scraper, washed with fresh CellGro® media and counted.

A sample of iDC at 1×10⁶ cells/ml was cultured for 18 h with a fixed amount of Example particle #1 or #2, Control particle #1 or #2, soluble peptide (SEQ ID NOs: 7, 8 or 9), or tumor cell lysate. As a negative control, a sample of iDCs at 1×10⁶ cells/ml was cultured with CellGro® media only. Each culture used CellGro® media supplemented with a maturation cocktail containing 20 ng/ml IL-4, 100 ng/ml GM-CSF, 1000 IU/ml IFNγ, 2.5 μg/ml R848, 20 μg/mL Poly I:C (Hiltonol) and 10 ng/ml LPS (see Lovgren et al., Cancer Immunol Immunother, 2017, 66:1333-1344).

Mature DCs were harvested from each culture, washed with fresh CellGro® media and resuspended in in fresh CellGro® media to provide the DC vaccines shown in Table 5.

TABLE 5
Details of DC vaccines prepared from healthy donor blood.

| | | Phagocytosable | Ratio of |
|---|---|---|---|
| DC | | particle/soluble | peptide/particle/cell |
| Vaccine ref: | Donor blood | peptide/cell lysate | lysate* to iDCs |
| DCV#1 | Healthy donor 1 | CellGro ® media only | N/A |
| DCV#2 | Healthy donor 1 | ETV6 neoantigen | 10 μg peptide: 1 × 10⁶ iDCs |
| | | (SEQ ID NO: 7) | |
| DCV#3 | Healthy donor 1 | NUP210 neoantigen | 10 μg peptide: 1 × 10⁶ iDCs |
| | | (SEQ ID NO: 8) | |
| DCV#4 | Healthy donor 1 | ANRU tumor cell lysate | 30 ng lysate: 1 × 10⁶ iDCs |
| DCV#5 | Healthy donor 1 | ANRU tumor cell lysate | 60 ng lysate: 1 × 10⁶ iDCs |
| DCV#6 | Healthy donor 1 | Control particle #1 | 40 particles: 1 iDC |
| DCV#7 | Healthy donor 1 | Example particle #1 | 10 particles: 1 iDC |
| DCV#8 | Healthy donor 1 | Example particle #1 | 20 particles: 1 iDC |
| DCV#9 | Healthy donor 1 | Example particle #1 | 40 particles: 1 iDC |
| DCV#10 | Healthy donor 2 | CellGro ® media only | N/A |
| DCV#11 | Healthy donor 2 | ETV6 neoantigen | 10 μg peptide: 1 × 10⁶ iDCs |
| | | (SEQ ID NO: 7) | |
| DCV#12 | Healthy donor 2 | NUP210 neoantigen | 10 μg peptide: 1 × 10⁶ iDCs |
| | | (SEQ ID NO: 8) | |
| DCV#13 | Healthy donor 2 | ANRU tumor cell lysate | 30 ng lysate: 1 × 10⁶ iDCs |
| DCV#14 | Healthy donor 2 | ANRU tumor cell lysate | 60 ng lysate: 1 × 10⁶ iDCs |
| DCV#15 | Healthy donor 2 | Control particle #1 | 40 particles: 1 iDC |
| DCV#16 | Healthy donor 2 | Example particle #1 | 10 particles: 1 iDC |
| DCV#17 | Healthy donor 2 | Example particle #1 | 20 particles: 1 iDC |
| DCV#18 | Healthy donor 2 | Example particle #1 | 40 particles: 1 iDC |
| DCV#19 | Healthy donor 3 | NUP210 wild-type | 10 μg peptide: 1 × 10⁶ iDCs |
| | | (SEQ ID NO: 9) | |
| DCV#20 | Healthy donor 3 | ANRU tumor cell lysate | 30 ng: 1 × 10⁶ iDCs |
| DCV#21 | Healthy donor 3 | Control particle #2 | 10 particles: 1 iDC |
| DCV#22 | Healthy donor 3 | Example particle #2 | 1 particle: 1 iDC |
| DCV#23 | Healthy donor 3 | Example particle #2 | 3 particles: 1 iDC |
| DCV#24 | Healthy donor 3 | Example particle #2 | 10 particles: 1 iDC |
| DCV#25 | Healthy donor 4 | CellGro ® media only | N/A |
| DCV#26 | Healthy donor 4 | ANRU tumor cell lysate | 30 ng lysate: 1 × 10⁶ iDCs |
| DCV#27 | Healthy donor 4 | Example particle #2 | 25 particles: 1 iDC |

*the amount of cell lysate shown in the table refer to the protein concentration of the cell lysate as measured by absorbance at 280 nm.

b) Preparation of a DC Vaccine Using Blood from Patient ANRU:

Monocytes and CD8+ T-cells were obtained by leukapheresis from patient ANRU. CD14+ cells were used to prepare DC vaccines according to the method described above in section a). Details of the DC vaccines are shown in Table 6.

TABLE 6
Details of DC vaccines prepared from patient ANRU blood.

| | | | Ratio of |
|---|---|---|---|
| DC | | | peptide/particle/cell |
| Vaccine | Donor blood | peptide/particle/cell lysate | lysate* to iDCs |
| DCV#28 | Patient ANRU | CellGro ® media only | N/A |
| DCV#29 | Patient ANRU | NUP210 neoantigen | 10 μg peptide: 1 × 10⁶ iDCs |
| | | (SEQ ID NO: 8) | |
| DCV#30 | Patient ANRU | ETV6 neoantigen | 10 μg peptide: 1 × 10⁶ iDCs |
| | | (SEQ ID NO: 7) | |
| DCV#31 | Patient ANRU | ANRU tumor cell lysate | 30 ng lysate: 1 × 10⁶ iDCs |
| DCV#32 | Patient ANRU | IFNγ ANRU tumor cell lysate^{†} | 30 ng lysate: 1 × 10⁶ iDCs |
| DCV#33 | Patient ANRU | Control Particle #1 | 40 particles: 1 iDC |
| DCV#34 | Patient ANRU | Example Particle #1 | 3 particles: 1 iDC |
| DCV#35 | Patient ANRU | Example Particle #1 | 20 particles: 1 iDC |
| DCV#36 | Patient ANRU | Example Particle #1 | 40 particles:1 iDC |

*the amount of cell lysate shown in the table refer to the protein concentration of the cell lysate as measured by absorbance at 280 nm.
^{†}IFNγ was added to the ANRU tumor cells used to prepare this lysate before lysis. IFNγ is known to upregulate the immunoproteasome and the MHCI antigen presenting machinery, thus increasing the number of peptides presented by the DCs via the MHCI.

c) Ex Vivo Expansion of Autologous T-Cells Using the DC Vaccines:

The DCs in each DC vaccine (DCV #1 to #36) were counted and placed in co-culture with the CD8⁺ T-cells isolated from autologous PBMCs isolated from the respective donor blood at a ratio of CD8⁺ T-cell to DCs of 1:5.

The co-cultures were incubated for 6 days in CellGro® media supplemented with 20 U/ml IL-2. At day 6, each co-culture was maintained by exchanging 50% of the medium or by splitting the cells. From day 6, the CellGro® media was supplemented with 300 U/ml IL-2.

At day 10 to 14, the expanded T-cells in each co-culture were harvested, washed and counted. The expanded T-cells were then exposed to ANLU tumor cells. The level of tumor specific activation of the expanded T-cells following exposure to ANLU tumor cells was assessed by flow cytometry by measuring the expression of CD107a and/or IFNγ, which are indicators of T-cell degranulation.

Results:

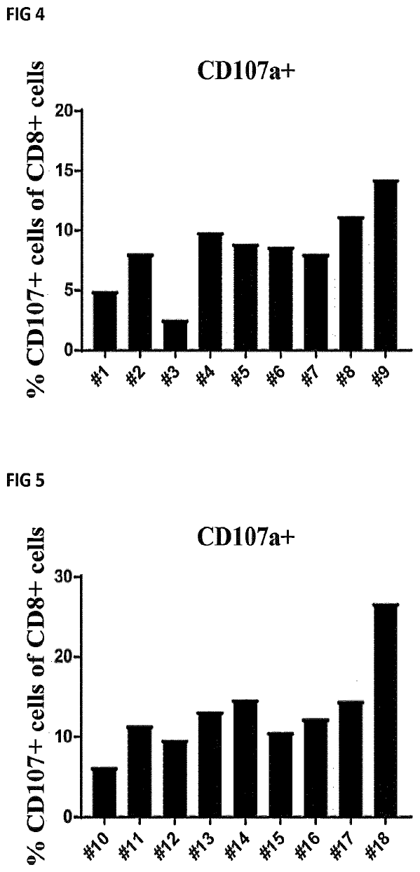

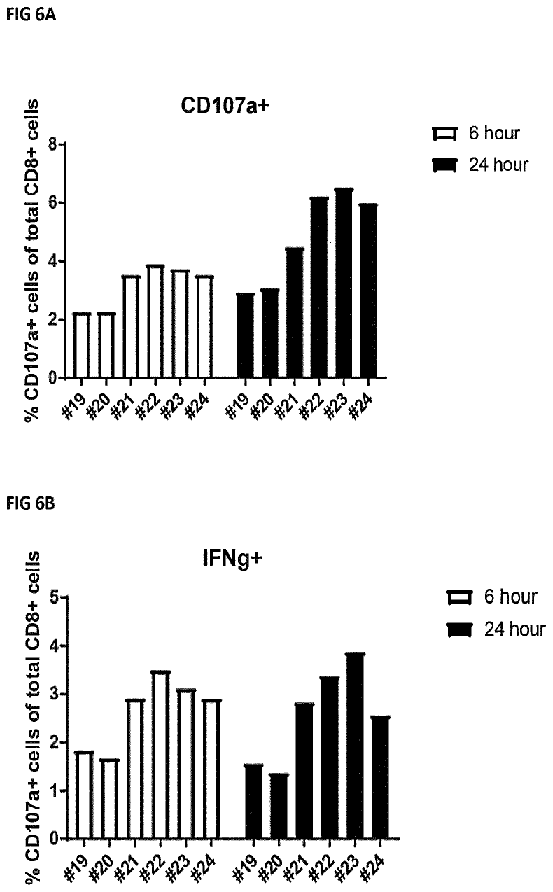

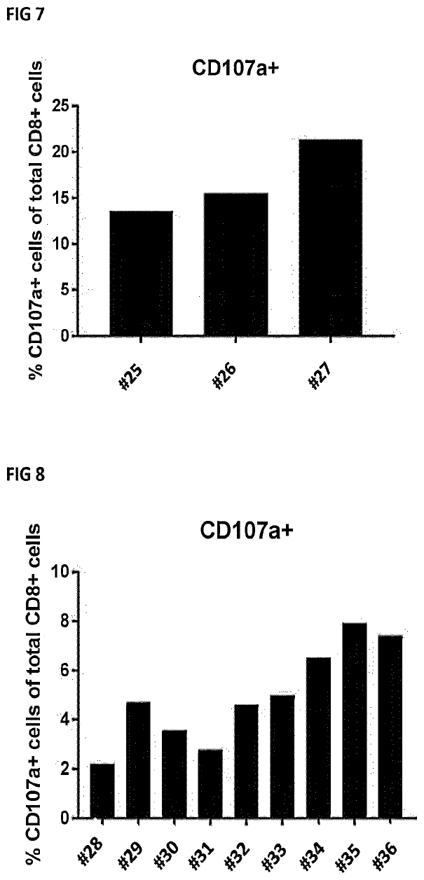

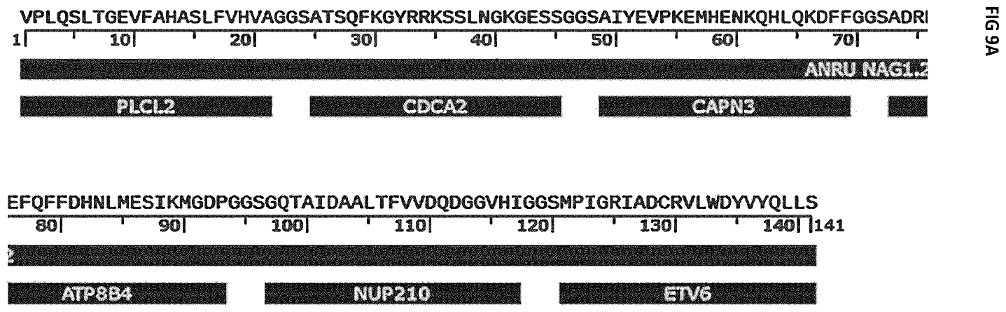

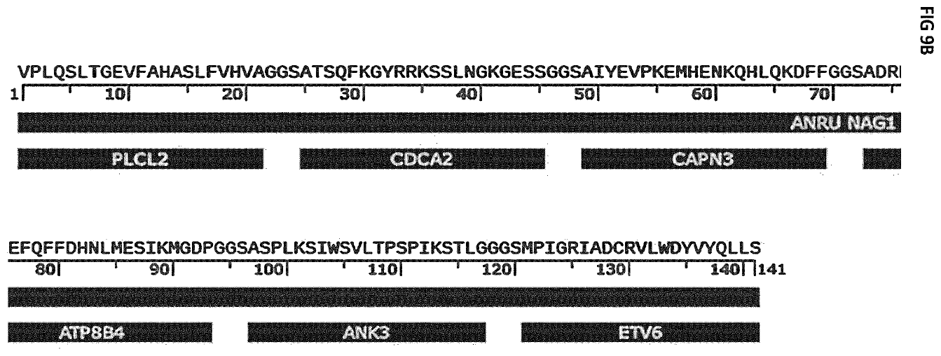

The ability of the DC vaccines to specifically activate and expand anticancer T-cells was assessed by expanding autologous T-cells in vitro using a DC vaccine, and then measuring the percentage of the expanded T-cells that recognized and were specifically activated by a tumor cell. Tumor specific T-cell activation was measured by monitoring T-cell degranulation by detecting CD107a and/or IFNγ expression by T-cells following exposure to tumor cells. The percentage of activated T-cells present in the expanded T-cell samples prepared using the DC vaccines are shown in FIGS. 4 to 8.

The T-cells expanded using a DC vaccine prepared using Example particles #1 or #2 (i.e. DCV #7-9, 16-18, 22-24 27 and 34-36) were found to express CD107a and/or IFNγ at a higher level than the T-cells expanded using a DC vaccine prepared with a soluble peptide (i.e. DCV #2, 3, 11, 12, 29 and 30) or tumor cell lysate (i.e. DCV #4, 5, 13, 14, 20, 26, 31 and 32). These data therefore show that DC cancer vaccines prepared using Example particles #1 or #2 were superior at specifically activating and expanding anticancer T-cells compared to DC vaccines prepared using soluble peptides or tumor cell lysates

### Example 7: Ex Vivo Expansion of Anticancer T-Cells from PBMCs Harvested from a Patient Previously Treated with a DC Cancer Vaccine of the Invention

Following administration of a DC cancer vaccine of the present invention to a patient, a blood sample is taken from the patient preferentially using a leukapheresis and an elutriation device. When whole blood is taken from the patient, the blood sample is processed to provide a PBMC sample. Using an elutriation device, a lymphocyte fraction with a very high frequency (^{˜}90%) of CD3+ cell population is obtained. The CD3+ population may be further refined to provide a CD3+/CD8+ T-cell population via selection, for example using bead isolation of the desired CD8+ T cell population. The CD3+ population or CD3/CD8+ T-cell population is placed in co-culture with mature DCs previously incubated with phagocytosable particles as described herein. The DCs have, for example, been incubated with phagocytosable particles at a cell to particle ratio of about 1:40. The T-cells are activated and expanded by the mature DCs. After a sufficient time, for example, 5 to 15 days, the expanded T-cells are harvested from the co-culture. The number of viable T-cells in the harvested sample is determined. The expanded T-cells are then injected into the patient.

1. An in vitro method for the manufacture of a dendritic cell (DC) cancer vaccine, said method comprising the steps of:
(i) providing a plurality of phagocytosable particles, wherein each phagocytosable particle comprises a core and an antigenic construct tightly associated to the core, wherein the antigenic construct comprises at least one epitope peptide having an amino acid sequence corresponding to an amino acid sequence of a part of a protein or peptide known or suspected to be expressed by a cancer cell in a subject;
(ii) providing a sample of DCs; and
(iii) contacting the sample of DCs with the plurality of phagocytosable particles in vitro and under conditions allowing for the phagocytosis of at least one phagocytosable particle by a DC.

2. The method of claim 1, wherein the sample of DCs is derived from the subject.

3. The method of claim 1 or 2, wherein the sample of DCs is derived from a blood sample harvested from the subject, preferably a peripheral blood mononuclear cell (PBMC) sample harvested from the subject.

4. The method of any preceding claim, wherein step (iii) further comprises contacting the sample of DCs with a PDL1 inhibitor.

5. The method of any preceding claim, wherein said method further comprises the steps of:
(v) removing extracellular phagocytosable particles from the sample of DCs; and/or
(vi) isolating at least one DC containing at least one phagocytosable particle from the sample of DCs in step (iii).

6. The method of claim 5, wherein step (v) comprises the positive selection of a DC containing at least one phagocytosable particle by means of a magnet or magnetic field.

7. The method of any preceding claim, wherein the cancer cell in the subject is a skin cancer cell, breast cancer cell, colon cancer cell, liver cancer cell, lung cancer cell, pancreatic cancer cell, prostate cancer cell, ovarian cancer cell, bladder cancer cell, cervical cancer cell, sarcoma cell, head-and-neck cancer cell or renal cancer cell.

8. The method of any preceding claim, wherein the plurality of phagocytosable particles of step (i) has been subjected to a sterilising wash resulting in a plurality of sterile or aseptic phagocytosable particles.

9. The method of any preceding claim, wherein the antigenic construct comprises two or more covalently linked epitope peptides.

10. The method of any preceding claim, wherein the antigenic construct comprises three or more covalently linked epitope peptides; for example three, four or five covalently linked epitope peptides.

11. The method of claim 9 or 10, wherein the covalently linked epitope peptides are covalently linked via a spacer moiety.

12. The method of claim 11, wherein the spacer moiety is a sequence of 1 to 15 amino acids, preferably 1 to 5 amino acids, and more preferably comprising the amino acid sequence VVR and/or the amino acid sequence GGS.

13. The method of any one of claims 9 to 12, wherein each of the covalently linked epitope peptides is 3 to 25 amino acids in length.

14. The method of any preceding claim, wherein at least one of the epitope peptides is a neoepitope peptide having an amino acid sequence corresponding to an amino acid sequence of a part of a protein or peptide known or suspected to be expressed by a cancer cell in the subject, wherein the part of the protein or peptide has at least one somatic mutated amino acid.

15. The method of any preceding claim, wherein the antigenic construct is covalently attached to the core.

16. The method of any preceding claim, wherein the phagocytosable particle comprises two or more different antigenic constructs tightly associated to the core, for example two, three, four or five antigenic constructs tightly associated to the core.

17. The method of claim 16, wherein each of the different antigenic constructs comprise different epitope peptide sequences or a different combination of epitope peptides.

18. The method of any preceding claim, wherein the phagocytosable particle has a largest dimension of less than 5.6 μm, preferably less than 4 μm, more preferably less than 3 μm, even more preferably from 0.5 to 2 μm, or most preferably about 1 μm.

19. The method of any preceding claim, wherein the phagocytosable particle has paramagnetic properties, preferably superparamagnetic properties.

20. The method of any preceding claim, wherein the core comprises a polymer, preferably polystyrene.

21. The method of any preceding claim, wherein the phagocytosable particle has paramagnetic properties, has a largest dimension of less than 5.6 μm, and the core of the phagocytosable particle comprises polystyrene.

22. A DC cancer vaccine produced by the method of any one of claims 1 to 20.

23. The DC cancer vaccine of claim 22 comprising a PDL1 inhibitor.

24. A DC cancer vaccine of claim 22 or 23 for use as a medicament.

25. A DC cancer vaccine of claim 22 or 23 for use in the treatment or prophylaxis of a cancer in a subject.

26. The DC cancer vaccine for use according to claim 25, wherein the cancer is skin cancer, breast cancer, colon cancer, liver cancer, lung cancer, pancreatic cancer, prostate cancer, ovarian cancer, bladder cancer, cervical cancer, sarcoma, head-and-neck cancer or renal cancer.

27. A method of treating or preventing cancer comprising a step of administering to a subject in need thereof the DC cancer vaccine of claim 22 or 23.

28. The method as claimed in claim 27, or use as claimed in any one of claims 24 to 26, further comprising the steps of:
a) harvesting anticancer T-cells from a blood sample from the subject;
b) expanding the anticancer T-cells in vitro; and
c) administering a therapeutic dose of the expanded anticancer T-cells to the subject; wherein steps a), b) and c) are performed before administering the DC cancer vaccine to the subject, and/or wherein steps a), b) and c) are performed after administering a DC cancer vaccine of the invention to the subject.

29. The method as claimed in claim 28, or use as claimed in claim 28, wherein step b) comprises the steps of:
ba) providing a phagocytosable particle as defined in any one of claims 1 to 21,
bb) providing an antigen-presenting cell (APC);
25 bc) contacting the phagocytosable particle with the APC from step bb) in vitro and under conditions allowing phagocytosis of the phagocytosable particle by the APC;
bd) providing an anticancer T-cell from step a) of claim 28;
be) contacting the anticancer T-cell harvested from the subject with the APC from step bc) in vitro and under conditions allowing for specific activation and expansion of anti-cancer T-cells in response to antigen presented by the APC.

30. The method as claimed in any one of claims 27 to 29, or use as claimed in any one of claims 24 to 26, 28 and 29, wherein the subject is one whom has previously been, or is simultaneously being, administered a dose of anticancer T-cells, preferably a therapeutic dose of anticancer T-cells.

31. The method as claimed in claim 30 wherein the dose of anticancer T-cells administered to the subject contains at least one anticancer T-cell that recognises an epitope presented on the surface of a DC contained in the DC cancer vaccine administered to the subject according to claim 27, or use as claimed in claim 30 wherein the dose of anticancer T-cells administered to the subject contains at least one anticancer T-cell that recognises an epitope presented on the surface of a DC contained in the DC cancer vaccine for use according to any one of claims 24 to 26.

32. The method as claimed in claim 30 or 31, or use as claimed in claim 30 or 31, wherein a cytokine is simultaneously, sequentially or separately administered to the subject, preferably the cytokine is IL2.

33. Use of a phagocytosable particle in the preparation of a DC cancer vaccine, wherein said phagocytosable particle comprises a core and an antigenic construct tightly associated to the core, wherein the antigenic construct comprises at least one epitope peptide having an amino acid sequence corresponding to an amino acid sequence of a part of a protein or peptide known or suspected to be expressed by a cancer cell in a subject.

34. A method for the in vitro expansion of anticancer T-cells, said method comprising the steps of:
ba) providing a phagocytosable particle comprising a core and an antigenic construct tightly associated to the core, wherein the antigenic construct comprises at least one epitope having an amino acid sequence corresponding to an amino acid sequence of a part of a protein or peptide known or suspected to be expressed by a cancer cell in a subject;
bb) providing an APC;
bc) contacting the phagocytosable particle with the APC from step bb) in vitro, and under conditions allowing phagocytosis of the phagocytosable particle by the APC;
bd) providing an anticancer T-cell sample harvested from a blood sample from the subject; and
be) contacting the anticancer T-cell sample with the APC from step bc) in vitro and under conditions allowing specific activation and expansion of anticancer T-cells in response to antigen presented by the APC;
wherein the subject is one whom has previously been administered the DC cancer vaccine of claim 22 or claim 23.

35. A method of treating or preventing cancer comprising a step of administering to a subject the anticancer T-cells provided by the method of claim 34.
